(19) United States
(12) Patent Application Publication
(10) Pub. No.: US 2024/0132957 A1
(43) Pub. Date: Apr. 25, 2024

(54) CELL LYSIS ASSAY FOR CELL-FREE DNA ANALYSIS

(71) Applicant: Natera, Inc., San Carlos, CA (US)

(72) Inventors: Aoy Tomita MITCHELL, Wauwatosa, WI (US); Karl STAMM, Brookfield, WI (US)

(73) Assignee: Natera, Inc., San Carlos, CA (US)

(21) Appl. No.: 18/376,355

(22) Filed: Oct. 2, 2023

Related U.S. Application Data
(63) Continuation of application No. 16/597,559, filed on Oct. 9, 2019.
(60) Provisional application No. 62/743,195, filed on Oct. 9, 2018.
(60) Provisional application No. 62/883,864, filed on Aug. 7, 2019.

(51) Int. Cl. C12Q 1/6876; C12Q 1/686

(52) U.S. Cl. CPC C12Q 1/6876; C12Q 1/686; C12Q 2600/158

(57) ABSTRACT

Provided herein are methods for determining levels of cell lysis in samples, particularly sampled in which amounts of non-self cell-free DNA may be determined.

### RELATED APPLICATIONS

This application claims priority to U.S. Provisional Application No. 62/743,195, filed Oct. 9, 2018 and to U.S. Provisional Application No. 62/883,864, filed Aug. 7, 2019, the entire contents of each of which are incorporated herein by reference.

### FIELD OF THE INVENTION

Provided herein are methods for determining levels of in vitro and/or ex vivo cell lysis in biological samples, such as from humans or animals, particularly, but not limited to, samples in which amounts of “self” and/or “non-self” cell-free DNA may be determined (e.g., from human or animal organ perfusate samples).

### BACKGROUND

Cell-free DNA (cfDNA) can be isolated from biological samples such as whole blood, plasma, serum, other body fluids (e.g., organ perfusate fluids) and can be analyzed for a variety of purposes, such as transplant monitoring including general assessments of in vivo tissue damage. However, cellular lysis, such as from white blood cells (WBCs) can occur during or after sample collection or processing and result in genomic DNA being released from those cells. This can result in additional DNA from the subject (self) being introduced and can result in the dilution of non-self, such as a transplant donor, fraction. Accordingly, methods for quantifying cell lysis in a sample are needed.

### SUMMARY

In one aspect, provided herein are methods for determining the levels of cell lysis in a sample. The methods comprise determining an amount of a long Alu fragment in the sample, determining an amount of a short Alu fragment in the sample, and determining a ratio of the amount of the long Alu fragment and the short Alu fragment. The ratio is indicative of the amount of cell lysis in the sample. Determining the amount of the long Alu fragment and the amount of the short Alu fragment may comprise amplification using a forward primer and a reverse primer for the long Alu fragment, a forward primer and a reverse primer for the short Alu fragment, and one or more probes. For example, amplifications may be performed using RT-qPCR.

In some aspects, the long Alu fragment may be ALU175, ALU224, ALU247, or ALU254. The short Alu fragment may be ALU115 or ALU79. In particular embodiments, the long fragment is ALU 247 and the short fragment is ALU115, and the calculated ratio is ALU247/ALU115.

In some aspects, the ratio is compared to a threshold value. A ratio greater than the threshold value may indicate that the sample is not suitable for analysis of non-self cell-free DNA in the sample. A ratio less than the threshold value indicates that the sample is suitable for analysis. In some aspects, the method further comprises determining the amount of non-self cell-free DNA in the sample. The methods may further comprise determining or suggesting a treatment regimen based on the determined amount of non-self cell-free DNA in the sample.

### BRIEF DESCRIPTION OF THE DRAWINGS

FIG. 1A and FIG. 1B show amplification curves for ALU 115 (FIG. 1A) and ALU247 (FIG. 1B) primers. Samples were run in triplicate following a standard TaqMan® protocol.

FIG. 2 shows ALU ratios across varying levels of cells spiked into plasma samples.

FIG. 3 shows a one-way analysis of ALU247/115 ratio and means comparisons for all pairs.

FIG. 4A shows the rat ALU247/115 ratio along with a graph depicting the raw ratio vs. box chronology.

FIG. 5A is a bar graph showing the ALU247/115 raw ratio along with quantile statistics.

FIG. 5B shows the summary statistics for the ALU24/115 ratio.

FIG. 6A is a graph showing the PC gDNA ALU ratio across 26 plates. FIG. 6B is a chart showing the gDNA ALU ratio limit summaries.

FIG. 7A is a graph showing the ALU115 efficiency across 26 plates. FIG. 7B is a chart showing ALU115 efficacy limit summaries.

FIG. 8A is a graph showing the ALU247 efficiency across 26 plates. FIG. 8B is a chart showing ALU247 efficiency limit summaries.

FIG. 9 shows the ALU ratio following various centrifuge speeds used during sample preparation.

FIG. 10 shows the total cf-DNA following various centrifuge speeds used during sample preparation.

FIG. 11 is a graphic showing a potential embodiment of the cell lysis assay described herein wherein ALU257 and ALU115 are measured.

FIG. 12A shows ALU247/115 ratios and calculated statistics for samples spiked with 0 cells and 2500 cells. FIG. 12B shows ALU247/115 ratios and calculated statistics for samples spiked with 5000 cells and 7500 cells.

FIG. 13A-13C shows the raw ALU247/ALU115 ratio in samples spiked with increasing amounts of cells (0, 2500, 5000, and 7500). In FIG. 13A, the LOD and LOB are indicated by the green and blue lines, respectively. In FIG. 13B, a best fit line is drawn to show the increasing ratio as the number of cells increases. FIG. 13C shows the mean ratio (raw ALU247/ALU115) in each sample.

FIG. 14 shows the false positive rate vs. the true positive rate for the ROC curve calculated to differentiate samples with 2500 or more cells lysed from samples with 0 cells lysed.

FIG. 15A shows the raw ALU247/ALU115 ratio across 36 samples. Limit summaries are shown in FIG. 15B.

FIG. 16 shows the ALU247/115 ratio for samples spiked with 2500 cells.

FIG. 17A shows exemplary primers and probes that may be used. ALU247 forward primer may be 5′-GTGGCTCACGCCTGTAATC-3′ (SEQ ID NO: 4), ALU115 forward primer may be 5′-CCTGAGGTCAGGAGTTCGAG-3′ (SEQ ID NO: 2), the ALU common probe may be 5′ CCAGCCTGGCCAACATGGTG 3′ (SEQ ID NO: 1), the ALU115 reverse primer may be TGTAATCCCAGCTACTCGGG (SEQ ID NO: 6), or the ALU247 reverse primer may be CCACTGCACTCCAGCCTG (SEQ ID NO: 7).

FIG. 17B shows exemplary primers and probes that may be used, for example to generate an ALU product of 224 base pairs in length (ALU224). The ALU224 forward primer may be CACTTTGGGAGGCCGAGG (SEQ ID NO: 8), the ALU115 forward primer may be CCTGAGGTCAGGAGTTCGAG (SEQ ID NO: 2), the ALU common probe may be 5′ CCAGCCTGGCCAACATGGTG 3′ (SEQ ID NO: 1), the ALU115 reverse primer may be TGTAATCCCAGCTACTCGGG (SEQ ID NO: 6), or the ALU224 reverse primer may be CCACTGCACTCCAGCCTG (SEQ ID NO: 7).

FIG. 17C shows exemplary primers and probes that may be used, for example to generate an ALU product of 254 base pairs in length (ALU254). The ALU254 forward primer may be GTGGCTCACGCCTGTAATC (SEQ ID NO: 4), the ALU115 forward primer may be CCTGAGGTCAGGAGTTCGAG (SEQ ID NO: 2), the ALU common probe may be CCAGCCTGGCCAACATGGTG (SEQ ID NO: 1), the ALU115 reverse primer may be TGTAATCCCAGCTACTCGGG (SEQ ID NO: 6), or the ALU254 reverse primer may be CCACTGCACTCCAGCCTGGGCGACA (SEQ ID NO: 9).

FIG. 18 shows measurements (by TAQMAN and SYBR) of ALU ratio, ALU247, and ALU115 from a collection of 77 human patient samples spiked with varying amounts of cells. ALU115 amount vs. cells spiked is shown in FIG. 19A and quantified in FIG. 19B. ALU247 amount vs. cells spiked is shown in FIG. 20A and quantified in FIG. B.

FIG. 21A-21C shows a comparison of the ALU ratio calculated by SYBR and TAQMAN. The samples with lower total input DNA as measured by the short fragment (first tertile TAQMAN ALU 115 grams) had a lower correlation (76% vs 92%) between SYBR and TAQMAN ALU Ratios than the samples with higher total input DNA as measured by the short fragment (third tertile TAQMAN ALU 115 grams) Samples in the lower third of overall DNA quantity by TAQMAN correlate by 75.6% and by SYBR 85.6% (FIG. 21A). Samples in the upper third of overall DNA quantity by either technology correlate by 92% (FIG. 21B). A greater difference in correlations indicates that the TAQMAN assay has more precision in the lower input samples than the SYBR assay (FIG. 21C). This indicates that this assay was more reliable with lower DNA inputs on the TAQMAN chemistry.

FIG. 22A-22C show ALU assays comparing SYBR vs. TAQMAN in contrived samples (cell lysis Experiment 2). SYBR cannot distinguish the low level of DNA addition from background, whereas TAQMAN short and long fragment assays have twice the sensitivity (fold-change) to DNA additions (FIG. 22A-22C).

FIG. 23 shows an example of a multiplexed ALU assay using primers for a 175 base pair long ALU fragment (ALU175) and a 79 base pair short ALU fragment (ALU79). Both products could be amplified in a single reaction with two probes labeled with two distinct dyes. For example, one probe may be labeled with FAM and one probe may be labeled with VIC. The ALU175 forward primer may be GTGGCTCACGCCTGTAATC (SEQ ID NO:4), a first ALU probe may be GTCAGGAGTTCGACCAGC (SEQ ID NO: 10), the ALU175 reverse primer may be AGCTACTCGGGAGGCTGAG (SEQ ID NO: 11), the ALU79 forward primer GCAGGAGAATCGCTTGAACC (SEQ ID NO: 12), a second ALU probe may be GGTTGCAGTGAGCCGAGAT (SEQ ID NO: 13), or the ALU79 reverse primer may be ACTCCAGCCTGGGCGACA (SEQ ID NO: 14).

FIG. 24A shows the raw ALU247/115 ratio in samples containing varying amounts of input DNA (in picograms). FIG. 24B shows the one-way analysis of raw 247/115 ratio for the data shown in FIG. 24A. Means comparisons are shown in FIG. 24C.

FIG. 25 shows an electropherogram image of sheared gDNA, a simulation of cfDNA. An Agilent 2100 Bioanalyzer instrument and high sensitivity DNA Kit were used to demonstrate the 164 by peak corresponding to the median distribution of gDNA sheared by ultrasonication to the size range of cfDNA of apoptotic origin. FU, fluorescence units; bp, base pairs. Peaks at 35 and 10380 bp represent lower and upper internal kit standards.

FIG. 26 is a graphic highlighting one embodiments of the ALU assay described herein, showing the location of the ALU247 and ALU115 primers and the TAQMAN probe, respectively. ALU115 primers amplify all ALU fragments greater than 115 base pairs in length, including 140-200 bp fragments typically produced by apoptosis. In contrast, ALU247 primers can only amplify longer fragments (greater than 247 bp), typically produced by necrosis and cell lysis.

FIG. 27A-27B show LoB Measurement Distributions for ALU115 for reagent lot A (FIG. 27A) and reagent lot B (FIG. 27B). LoB, calculated according to the CLSI nonparametric option [1] and denoted by the dashed vertical line is 0.014 pg/μl.

FIG. 28A-28B show LoB Measurement Distributions for ALU 247 for reagent lot A (FIG. 28A) and reagent lot B (FIG. 28B). LoB, calculated according to the CLSI nonparametric option [1] and denoted by the dashed vertical line is 0.006 pg/μl.

FIG. 29A-29B show DNA Fragmentation Assay Linearity Results. FIG. 29A shows results for ALU115 and FIG. 29B show results for ALU247.

FIG. 30A-30C show interfering substance results at 2 ng/ml cfDNA for the DNA Fragmentation Assay. FIG. 30A shows one-way Analysis of Alu ratio. Results are quantified in FIG. 30B. The Connecting Letters Report is shown in FIG. 30C. At this TCF concentration, statistically significant, but not clinically significant, differences compared to the unspiked control were seen for Sirolimus, EDTA, and bilirubin.

FIG. 31A-31B show interfering substance results at 25 ng/ml cfDNA for the DNA fragmentation assay. Results are shown in FIG. 31A and quantified in FIG. 31B.

FIG. 32A-C show interfering substance results at 50 ng/ml cfDNA for the DNA Fragmentation Assay. FIG. 32A shows one-way Analysis of Alu ratio. Results are quantified in FIG. 32B. The Connecting Letters Report is shown in FIG. 32C.

FIG. 33A-33B show quantitative effects of leukocyte lysis on ALU247/115 ratio (FIG. 33A) and mean donor fraction (FIG. 33B).

FIG. 34A-34B show mathematical modeling of effect of adding leukocyte gDNA on apparent donor fraction (DF).

FIG. 35A-B show bioanalyzer electropherograms of patient plasma cfDNA samples. Patient sample collected and processed per TAI protocol shows predominant singlet and doublet apoptotic cfDNA peaks at 186 bp and 362 bp, respectively, without larger fragments produced by cellular lysis (FIG. 35A). Human sample procured and processed by a commercial vendor with delayed centrifugation (>24 hrs) shows a small peak at 178 bp (probably apoptotic) and a large, broad peak centered at 7822 bp, consistent with origin from leukocyte lysis (FIG. 35B). In both figures, sharp peaks at 35 bp and 10380 bp are internal kit markers.

FIG. 36A-36B shows a comparison of ALU247/115 ratios for samples collecting in BCT tubes, EDTA tubes, and PPT tubes from four different donors. Streck BCT tubes, EDTA tubes, and BD Bioscience's PPT tubes were compared.

FIG. 37A-37B shows a comparison of ALU247/115 ratios measured using SYBR for samples containing 50 pg input DNA. Biomatrica BIM cell preservation tubes and BD Bioscience's PPT tubes were compared. Results are shown in FIG. 37A and quantified in FIG. 37B.

FIG. 38A shows a comparison of normalized ALU257/115 ratios in the Streck cell preservation tube (BCT) against the PPT tube and a modified tube (BCT->PPT tube). Results are quantified in FIG. 38B.

FIG. 39A shows a comparison of ALU247/115 ratios measured by SYBR for samples containing 50-70 pg input DNA in BIM tubes, PPT tubes, and a modified tube. Results are quantified in FIG. 39B.

### DETAILED DESCRIPTION

Cell lysis, such as from mechanical stress and degenerative changes after sample collection, as opposed to apoptosis, releases long genomic fragments in biological samples, such as blood samples. On the other hand, the majority of cfDNA from blood drawn from normal healthy individuals appears to be the result of normal cellular apoptosis and turnover. Apoptosis results in the release of relatively short DNA fragments, significantly shorter than those released by cellular lysis. Samples in which significant cell lysis has occurred may not be suitable for analysis of amounts of non-self cell-free DNA. In contrast, samples in which lower levels of (or no) cell lysis has occurred may be suitable for analysis of amounts of non-self DNA.

An Alu element is a short stretch of DNA originally characterized by the action of the Arthrobacter luteus (Alu) restriction endonuclease. Alu repeats are the most abundant sequences in the human genome, with a copy number of about 1.4 million per genome. Alu sequences are short interspersed nucleotide elements (SINEs), typically 300 nucleotides, which account for more than 10% of the genome. Alu DNA sequences are particularly useful when working with low amounts of cfDNA. For example, this high copy number makes them advantageous targets for highly sensitive detection and DNA fragmentation analysis of low template populations such as circulating cfDNA in organ transplant patients.

Provided herein are methods, referred to herein as DNA fragmentation assays, for measuring the potential contaminating contribution of cell lysis of a cfDNA sample by analyzing long Alu fragments versus short Alu fragments in the sample. As used herein, a “long fragment” refers to an Alu fragment that is greater than 170 bps (e.g., between 171 and 300 bps in length), while a “short fragment” is an Alu fragment that is less than or equal to 170 bps (e.g., between 75 and 170 bps in length). The methods may comprise determining an amount of a long Alu fragment in the sample, determining an amount of a short Alu fragment in the sample, and determining the ratio of the amount of the long Alu fragment and the short Alu fragment. The ratio is indicative of the amount of cell lysis in the sample.

Any suitable short Alu fragment may be measured. The short Alu fragment may be any suitable size of less than or equal to 170 bps in length. For example, the short Alu fragment may be between 75 and 170 bps in length. For example, the short Alu fragment may be 170 bps in length, less than 170 bps in length, less than 160 bps in length, less than 150 bps in length, less than 140 bps in length, less than 130 bps in length, less than 120 bps in length, less than 110 bps in length, less than 100 bps in length, less than 90 bps in length, less than 80 bps in length, or 75 bps in length. In some embodiments, the short Alu fragment may be ALU 115, which is produced by amplification of both long and short fragment Alu in the sample. Accordingly, in some embodiments, the methods provided herein can be used to identify and quantify cell lysis on the basis of the ratio of long Alu fragments (such as those represented by the ALU 247 amplicon) to the total amount of Alu fragments (e.g. both short and long fragments, such as those represented by the ALU 115 amplicon) in a sample. In some embodiments, the short Alu fragment may be ALU79.

Any suitable long Alu fragment may be measured. The Alu fragment should be a long enough fragment to differentiate between fragments typically produced by apoptosis vs. fragments produced by non-apoptotic cell death, such as those produced by cell lysis. In some embodiments, the long Alu fragment is greater than 170 bps, greater than 180 bps, greater than 190 bps, greater than 200 bps, greater than 210 bps, greater than 220 bps, greater than 230 bps, greater than 240 bps, greater than 250 bps, greater than 260 bps, greater than 270 bps, greater than 280 bps, greater than 290 bps, or 300 bps in length. In some embodiments, the long Alu fragment may be ALU175. In some embodiments, the long Alu fragment may be ALU224. In some embodiments, the long Alu fragment may be ALU247. In some embodiments, the long Alu fragment may be ALU254.

The methods described herein are generally performed with primers differentially targeting long Alu fragment and short Alu fragment to determine a ratio. The assays described herein may be performed with any suitable primers and one or more probes to detect any desired combination of long and short Alu fragments in the sample. For example, primers to detect a long Alu fragment (such as ALU175, ALU224, ALU247, or ALU254) can be used in combination with primers to detect a short Alu fragment (such as ALU115, or ALU79) to determine the ratio of long Alu: short Alu in the sample. In some embodiments, primers for long Alu fragment amplifying a 247 bp length of Alu sequence can be used in combination with primers for short Alu fragment amplifying a 115 bp length of Alu sequence. In such an embodiment the ratio is an ALU247/ALU115 ratio.

In some embodiments, the assays provided herein are superior to standard methods for analyzing cell lysis because they can be performed with picogram amounts of cfDNA. In contrast, existing methods to detect cell lysis require 100-1000 times more cells or 10-fold more DNA. In any one of the methods provided herein the samples can have as little as 20 pg cfDNA. In any one of the methods provided herein the samples can have as little as 20 pg, 30 pg, 40 pg, 50 μg, 60 pg, 70 pg, 80 pg, 90 pg, 100 pg, 110 pg, 120 pg, 130 pg, 140 pg, 150 μg, 175 pg, 200 pg, or 250 pg cfDNA. In any one of the methods the samples have no more than 200 pg, 175 pg, 150 μg, 140 pg, 130 pg, 120 pg, 110 pg or 100 pg input DNA. The input DNA for any one of the samples provided herein in any one of the methods provided herein can have a combination of any one of the lower limits provided herein in combination with any one of the upper limits provided herein as the combination is an appropriate combination where the lower limit and upper limit have a range and are thus applicable to each other.

In some embodiments, the methods provided herein may comprise determining an amount of a long Alu fragment in the sample, determining the total amount of Alu fragments in the sample (e.g. both short and long fragments), and determining the ratio of the amount of the long Alu fragment to the total amount of Alu fragments in the sample. The ratio is indicative of the amount of cell lysis in the sample. For example, the long Alu fragment may be measured using primers to detect ALU175, ALU224, ALU247, or ALU254 and the total amount of Alu in the sample can be measured by using primers to detect ALU115, which is produced by amplification of both long and short fragment Alu in the sample.

The DNA fragmentation assay described herein may be used to quantify cell lysis effectively even in the presence of various potentially interfering substances within the sample. For example, the DNA fragmentation assays may be used to quantify cell lysis in a plasma sample containing endogenous substances commonly elevated in plasma samples from heart transplant recipients. Additionally, the DNA fragmentation assays may be used to quantify cell lysis in the presence of exogenous substances commonly introduced into the plasma of transplant patients as a result of standard medical therapies. For example, the sample may contain one or more substances including bilirubin, hemoglobin, EDTA, prednisone, tacrolimus, sirolimus, mycophenolate, cyclosporine A, triglycerides, and IVIg, viruses (e.g. CMV, BKV).

As used herein, ALU 115 is an amplicon obtainable by amplifying the ALU repeats in the human genome using a forward primer, such as 5′-CCTGAGGTCAGGAGTTCGAG-3′ (SEQ ID NO: 2) and a reverse primer, such as 5′-CCCGAGTAGCTGGGATTACA-3′ (SEQ ID NO: 3). ALU 115 has a size of 115 base pairs. It is produced by amplification of both short and long fragment Alu.

As used herein ALU79 is an amplicon obtainable by amplifying the ALU repeats in the human genome using a forward primer, such as 5′-GCAGGAGAATCGCTTGAACC-3′ (SEQ ID NO: 12) and a reverse primer, such as 5′-ACTCCAGCCTGGGCGACA-3′ (SEQ ID NO: 14).

As used herein, ALU175 is an amplicon obtainable by amplifying the ALU repeats in the human genome using a forward primer, such as 5′-GTGGCTCACGCCTGTAATC-3′ (SEQ ID NO:4) and a reverse primer, such as 5′-AGCTACTCGGGAGGCTGAG-3′ (SEQ ID NO: 11). It is produced by amplification of long fragment Alu, but not short fragment Alu.

As used herein, ALU247 is an amplicon obtainable by amplifying the ALU repeats in the human genome using a forward primer, such as 5′-GTGGCTCACGCCTGTAATC-3′ (SEQ ID NO: 4) and a reverse primer, such as 5′-CAGGCTGGAGTGCAGTGG-3′ (SEQ ID NO:5). ALU 247 has a size of 247 base pairs. It is produced by amplification of long fragment Alu, but not short fragment Alu.

As used herein, ALU254 is an amplicon obtainable by amplifying the ALU repeats in the human genome using a forward primer, such as 5′-GTGGCTCACGCCTGTAATC-3′ (SEQ ID NO: 4) and a reverse primer, such as 5′-CCACTGCACTCCAGCCTGGGCGACA-3′ (SEQ ID NO: 9). It is produced by amplification of long fragment Alu, but not short fragment Alu.

As used herein, ALU224 is an amplicon obtainable by amplifying the ALU repeats in the human genome using a forward primer, such as 5′-CACTTTGGGAGGCCGAGG-3′ (SEQ ID NO: 8) and a reverse primer, such as 5′-CCACTGCACTCCAGCCTG-3′ (SEQ ID NO: 7). It is produced by amplification of long fragment Alu, but not short fragment Alu.

Additionally, the assays may be performed using one or more probes. In some embodiments, the assays are performed using a common Alu probe, such as 5′ CCAGCCTGGCCAACATGGTG 3′ (SEQ ID NO: 1).

In some aspects, multiplex assays may be performed. For example, a multiplex assay may be performed using primers for a long ALU fragment, a short ALU fragment, and two or more probes. Each of the two or more probes may be labeled with distinct dyes. Any suitable dyes may be used. For example, a multiplexed ALU assay may be performed using primers for a long Alu fragment and a short Alu fragment. Both products could be amplified in a single reaction with two probes labeled with two distinct dyes. For example, one probe may be labeled with FAM and one probe may be labeled with VIC.

In some embodiments, a multiplex assay could be performed to amplify a 175 base pair long ALU fragment (ALU175) and a 79 base pair short ALU fragment (ALU79). The ALU175 forward primer may be GTGGCTCACGCCTGTAATC (SEQ ID NO:4), a first ALU probe may be GTCAGGAGTTCGACCAGC (SEQ ID NO: 10), the ALU175 reverse primer may be AGCTACTCGGGAGGCTGAG (SEQ ID NO: 11), the ALU79 forward primer GCAGGAGAATCGCTTGAACC (SEQ ID NO: 12), a second ALU probe may be GGTTGCAGTGAGCCGAGAT (SEQ ID NO: 13), or the ALU79 reverse primer may be ACTCCAGCCTGGGCGACA (SEQ ID NO: 14).

In some embodiments, the methods further comprise spiking the sample with a cell standard of at least 250 cells. For example, the cell standard may be at least 500 cells, at least 1000 cells, at least 2500 cells, or at least 5000 cells.

As used herein, subjects have “self-specific” cell-free DNA (cfDNA) released into the blood stream by their own cells as a result of normal cellular turnover. In subjects, such as transplant recipients, a type of “non-self” (“donor specific” cfDNA in this instance) is also present in addition to “self-specific” cfDNA.

“Subject,” as used herein, refers to a human or animal, including all vertebrates, e.g., mammals such as primates (particularly higher primates), sheep, dog, rodents (e.g., mouse or rat), guinea pig, goat, pig, cat, rabbit, and cow, etc. In a preferred embodiment, the subject is a human. In a further preferred embodiment, the subject is a recipient of a transplant. In some embodiments, the subject is a child (e.g. under 18 years of age). In some embodiments, the subject is an infant (e.g., under 2 years of age). As used herein, “transplant” refers to the moving of an organ, tissue or portion thereof from a donor to a recipient for the purpose of replacing the recipient's damaged or absent organ, tissue or portion thereof. Any one of the methods or compositions provided herein may be used on a sample from a subject that has undergone a transplant of an organ or tissue. In some embodiments, the transplant is a heart transplant. In another embodiment, the subject is an experimental animal or animal suitable as a disease model.

Reports with any one or more of the values as provided herein are also provided in an aspect. Reports may be in oral, written (or hard copy) or electronic form, such as in a form that can be visualized or displayed. Preferably, the report provides the amount of cell lysis and/or non-self cfDNA in a sample. In some embodiments, the report provides amounts such as the aforementioned amounts in samples from a subject over time, and can further include corresponding threshold values in some embodiments.

In some embodiments, the amounts and/or threshold values are in or entered into a database. In some aspects, a database with such amounts and/or values is provided. From the amount(s), a clinician may assess the need for a treatment or monitoring of a subject. Accordingly, in any one of the methods provided herein, the method can include assessing a sample from the subject at more than one point in time. Such assessing can be performed with any one of the methods or compositions provided herein.

As used herein, “amount” refers to any quantitative value for the measurement as provided herein and can be given in an absolute or relative amount. Further, the amount can be a total amount, frequency, ratio, percentage, etc. As used herein, the term “level” can be used instead of “amount” but is intended to refer to the same types of values.

In some embodiments, any one of the methods provided herein can comprise comparing an amount to a threshold value. “Threshold” or “threshold value”, as used herein, refers to any predetermined level or range of levels that is indicative of something, such as the amount of cell lysis. The threshold value can take a variety of forms. It can be a single cut-off value, such as a median or mean. It can be established based upon comparative groups. It can be a range, for example. As another example, a threshold value can be determined from baseline values. Appropriate ranges and categories can be selected with no more than routine experimentation by those of ordinary skill in the art. The threshold value of any one of the methods, reports, databases, etc. provided herein, can be any one of the threshold values provided herein, such as in the Examples or Figures.

In some embodiments, the methods comprise comparing the ratio of the amount of the long Alu fragment and the short Alu fragment to a threshold value to determine whether the sample is suitable for analysis of non-self cfDNA. In some embodiments, a ratio above the threshold value indicates that the sample is not suitable for analysis of non-self cfDNA. The threshold value may be above 0.3. For example, the threshold value may be above 0.3, above 0.35, above 0.4, or above 0.5.

In some embodiments of any one of the methods provided herein the PCR is quantitative PCR meaning that amounts of nucleic acids can be determined. Quantitative PCR include real-time PCR, digital PCR, TAQMAN™, etc. In some embodiments of any one of the methods provided herein the PCR is “real-time PCR”. Such PCR refers to a PCR reaction where the reaction kinetics can be monitored in the liquid phase while the amplification process is still proceeding. In contrast to conventional PCR, real-time PCR offers the ability to simultaneously detect or quantify in an amplification reaction in real time. Based on the increase of the fluorescence intensity from a specific dye, the concentration of the target can be determined even before the amplification reaches its plateau.

The use of multiple probes can expand the capability of single-probe real-time PCR. Multiplex real-time PCR uses multiple probe-based assays, in which each assay can have a specific probe labeled with a unique fluorescent dye, resulting in different observed colors for each assay. Real-time PCR instruments can discriminate between the fluorescence generated from different dyes. Different probes can be labeled with different dyes that each have unique emission spectra. Spectral signals are collected with discrete optics, passed through a series of filter sets, and collected by an array of detectors. Spectral overlap between dyes may be corrected by using pure dye spectra to deconvolute the experimental data by matrix algebra.

A probe may be useful for methods of the present disclosure, particularly for those methods that include a quantification step. Any one of the methods provided herein can include the use of a probe in the performance of the PCR assay(s), while any one of the compositions or kits provided herein can include one or more probes.

As an example, a TAQMAN™ probe is a hydrolysis probe that has a dye label (e.g., FAM™ or VIC®) on the 5′ end, and minor groove binder (MGB) non-fluorescent quencher (NFQ) on the 3′ end. The TAQMAN™ probe principle generally relies on the 5″-3″ exonuclease activity of Taq® polymerase to cleave the dual-labeled TAQMAN™ probe during hybridization to a complementary probe-binding region and fluorophore-based detection. TAQMAN™ probes can increase the specificity of detection in quantitative measurements during the exponential stages of a quantitative PCR reaction.

PCR systems generally rely upon the detection and quantitation of fluorescent dyes or reporters, the signal of which increase in direct proportion to the amount of PCR product in a reaction. For example, in the simplest and most economical format, that reporter can be the double-stranded DNA-specific dye SYBR® Green (Molecular Probes). SYBR® Green is a dye that binds the minor groove of double-stranded DNA. When SYBR® Green dye binds to a double-stranded DNA, the fluorescence intensity increases. As more double-stranded amplicons are produced, SYBR® Green dye signal will increase.

It should be appreciated that the PCR conditions provided herein may be modified or optimized to work in accordance with any one of the methods described herein. Typically, the PCR conditions are based on the enzyme used, the target template, and/or the primers. In some embodiments, one or more components of the PCR reaction is modified or optimized. Non-limiting examples of the components of a PCR reaction that may be optimized include the template DNA, the primers (e.g., forward primers and reverse primers), the deoxynucleotides (dNTPs), the polymerase, the magnesium concentration, the buffer, the probe (e.g., when performing real-time PCR), and the reaction volume.

In any of the foregoing embodiments, any DNA polymerase (enzyme that catalyzes polymerization of DNA nucleotides into a DNA strand) may be utilized, including thermostable polymerases. Suitable polymerase enzymes will be known to those skilled in the art, and include E. coli DNA polymerase, Klenow fragment of E. coli DNA polymerase I, T7 DNA polymerase, T4 DNA polymerase, T5 DNA polymerase, Klenow class polymerases, Taq polymerase, Pfu DNA polymerase, Vent polymerase, bacteriophage 29, REDTaq™ Genomic DNA polymerase, or sequenase. Exemplary polymerases include, but are not limited to Bacillus stearothermophilus pol I, Thermus aquaticus (Taq) pol I, Pyrccoccus furiosus (Pfu), Pyrococcus woesei (Pwo), Thermus flavus (Tfl), Thermus thermophilus (Tth), Thermus litoris (Tli) and Thermotoga maritime (Tma). These enzymes, modified versions of these enzymes, and combination of enzymes, are commercially available from vendors including Roche, Invitrogen, Qiagen, Stratagene, and Applied Biosystems. Representative enzymes include PHUSION® (New England Biolabs, Ipswich, MA), Hot MasterTaq™ (Eppendorf), PHUSION® Mpx (Finnzymes), PyroStart® (Fermentas), KOD (EMD Biosciences), Z-Taq (TAKARA), and CS3AC/LA (KlenTaq, University City, MO).

Salts and buffers include those familiar to those skilled in the art, including those comprising MgCl2, Mg2(SO4), and Tris-HCl and KCl, respectively. Typically, 1.5-2.0 nM of magnesium is optimal for Taq DNA polymerase, however, the optimal magnesium concentration may depend on template, buffer, DNA and dNTPs as each has the potential to chelate magnesium. If the concentration of magnesium [Mg2+] is too low, a PCR product may not form. If the concentration of magnesium [Mg2+] is too high, undesired PCR products may be seen. In some embodiments the magnesium concentration may be optimized by supplementing magnesium concentration in 0.1 mM or 0.5 mM increments up to about 5 mM.

Buffers used in accordance with the disclosure may contain additives such as surfactants, dimethyl sulfoxide (DMSO), glycerol, bovine serum albumin (BSA) and polyethylene glycol (PEG), as well as others familiar to those skilled in the art. Nucleotides are generally deoxyribonucleoside triphosphates, such as deoxyadenosine triphosphate (dATP), deoxycytidine triphosphate (dCTP), deoxyguanosine triphosphate (dGTP), and deoxythymidine triphosphate (dTTP), which are also added to a reaction in an adequate amount for amplification of the target nucleic acid. In some embodiments, the concentration of one or more dNTPs (e.g., dATP, dCTP, dGTP, dTTP) is from about 10 μM to about 500 μM which may depend on the length and number of PCR products produced in a PCR reaction.

In some embodiments, the concentration of primers used in the PCR reaction may be modified or optimized. In some embodiments, the concentration of a primer (e.g., a forward or reverse primer) in a PCR reaction may be, for example, about 0.05 μM to about 1 μM. In particular embodiments, the concentration of each primer is about 1 nM to about 1 μM. It should be appreciated that the primers in accordance with the disclosure may be used at the same or different concentrations in a PCR reaction. For example, the forward primer of a primer pair may be used at a concentration of 0.5 μM and the reverse primer of the primer pair may be used at 0.1 μM. The concentration of the primer may be based on factors including, but not limited to, primer length, GC content, purity, mismatches with the target DNA or likelihood of forming primer dimers.

In some embodiments, the thermal profile of the PCR reaction is modified or optimized. Non-limiting examples of PCR thermal profile modifications include denaturation temperature and duration, annealing temperature and duration and extension time.

The temperature of the PCR reaction solutions may be sequentially cycled between a denaturing state, an annealing state, and an extension state for a predetermined number of cycles. The actual times and temperatures can be enzyme, primer, and target dependent. For any given reaction, denaturing states can range in certain embodiments from about 70° C. to about 100° C. In addition, the annealing temperature and time can influence the specificity and efficiency of primer binding to a particular locus within a target nucleic acid and may be important for particular PCR reactions. For any given reaction, annealing states can range in certain embodiments from about 20° C. to about 75° C. In some embodiments, the annealing state can be from about 46° C. to 64° C. In certain embodiments, the annealing state can be performed at room temperature (e.g., from about 20° C. to about 25° C.).

Extension temperature and time may also impact the allele product yield. For a given enzyme, extension states can range in certain embodiments from about 60° C. to about 75° C.

Quantification of the amounts from a PCR assay can be performed as provided herein or as otherwise would be apparent to one of ordinary skill in the art. As an example, amplification traces are analyzed for consistency and robust quantification. Internal standards may be used to translate the cycle threshold to amount of input nucleic acids (e.g., DNA).

Any one of the samples in any one of the methods provided herein may be collected in a tube, and one or more steps for doing so many be comprised in such methods. Such tubes include a Cell-Free DNA BCT®1 Streck Tube (cell preservation tube), a Biomatrica LBgard Blood Tube (cell preservation tube), or an EDTA-based BD Vacutainer® PPT™ Plasma Preparation Tube (physical separator tube).

Any one of the methods provided herein can also comprise extracting nucleic acids, such as cell-free DNA, from the sample. Such extraction can be done using any method known in the art or as otherwise provided herein (see, e.g., Current Protocols in Molecular Biology, latest edition, or the QIAamp Circulating Nucleic Acid kit or other appropriate commercially available kits). An exemplary method for isolating cell-free DNA from blood is described. Blood containing an anti-coagulant such as EDTA or DTA is collected from a subject. The plasma, which contains cfDNA, is separated from cells present in the blood (e.g., by centrifugation or filtering). An optional secondary separation may be performed to remove any remaining cells from the plasma (e.g., a second centrifugation or filtering step). The cfDNA can then be extracted using any method known in the art, e.g., using a commercial kit such as those produced by Qiagen. Other exemplary methods for extracting cfDNA are also known in the art (see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47:1607-1613; and Swinkels et al. Effects of Blood-Processing Protocols on Cell-free DNA Quantification in Plasma. Clinical Chemistry, 2003, vol. 49, no. 3, 525-526).

In one aspect, a method provided herein can include steps for determining amounts of non-self cfDNA, such as the donor fraction (DF) of cfDNA. These amounts can be determined using any methods provided herein or otherwise known in the art. In some embodiments, any one of the methods for determining cfDNA may be any one of the methods of U.S. Publication No. 2015-0086477-A1, and such methods are incorporated herein by reference in their entirety. An amount of cfDNA may also be determined by a MOMA assay. In some embodiments, any one of the methods for determining cfDNA may be any one of the methods of PCT Publication No. WO 2016/176662 A1, and such methods are incorporated herein by reference in their entirety.

In some embodiments, the Alu fragmentation assays described herein may be used to improve accuracy of determining the estimated portion of non-self cfDNA in a sample. For example, the amount of cell lysis in the sample, as quantified by the methods described herein, may be applied to a donor fraction correction formula to improve accuracy of the estimated fraction of cfDNA that is donor derived. For example, the amount of donor-derived cell free DNA in the sample may be subjected to a correction formula. The correction formula may be a calculation as shown in formula 1, wherein the observed donor fraction of cfDNA is multiplied by a correction factor (CF) and divided by 1-the ALU ratio as calculated by the methods described herein. This calculation is shown in formula 1:

$$Corrected D F = \frac{( Observed D F ) * C F}{1 - Alu ratio} \qquad Formula 1$$

The correction factor may be empirically determined, and may vary depending on the sample collection and processing protocol used. For example, the correction factor may vary depending on the tube that was used to collect, ship, and/or store the cf-DNA sample. For plasma collected in PPT tubes, the correction ratio may be about 0.7. Such corrections would be useful in any circumstance in which cfDNA samples are processed and shipped to improve accuracy of the estimated donor fraction of cell free DNA in the sample.

In some embodiments, samples with a high degree of cell lysis (e.g. above a threshold value) may be still be used for analysis of non-self portion of cfDNA in the sample using a correction factor as described herein. Accordingly, applying a correction factor to improve accuracy of the calculated amount of non-self portion of cfDNA may enhance the ability to use samples even when sample processing, storage, and handling causes significant cell lysis.

In some embodiments, the amounts of non-self DNA may be used to determine monitoring or treatment methods that should be used in the subject. Any one of the methods provided herein can include steps for doing so.

“Determining a monitoring regimen”, as used herein, refers to determining a course of action to monitor a condition in the subject over time. In some embodiments of any one of the methods provided herein, determining a monitoring regimen includes determining an appropriate course of action for determining the amount of non-self cfDNA in a subject over time or at a subsequent point in time, or suggesting such monitoring to the subject. This can allow for the measurement of variations in a clinical state and/or permit calculation of normal values or baseline levels (as well as comparisons thereto). In some embodiments of any one of the methods provided herein determining a monitoring regimen includes determining the timing and/or frequency of obtaining samples from the subject and/or determining or obtaining an amount of non-self cfDNA.

“Determining a treatment regimen”, as used herein, refers to the determination of a course of action for treatment of a subject. In some embodiments of any one of the methods provided herein, determining a treatment regimen includes determining an appropriate therapy or information regarding an appropriate therapy to provide to a subject, and any one of the methods provided herein can include such step(s). In some embodiments of any one of the methods provided herein, the determining includes providing an appropriate therapy or information regarding an appropriate therapy to a subject.

As used herein, information regarding a treatment or therapy or monitoring may be provided in written form or electronic form. In some embodiments, the information may be provided as computer-readable instructions. In some embodiments, the information may be provided orally.

The methods provided can include the step of providing a therapy, such as an anti-rejection therapy, or providing information regarding therapies, to the subject following a determination of the amount of non-self cfDNA in the sample. For example, therapy may be provided to the subject when the amount of non-self cfDNA in the sample is determined to be above a threshold value, such as 1%. In some embodiments, the information includes written materials containing the information. Written materials can include the written information in electronic form.

Therapies can include anti-rejection therapies. Anti-rejection therapies include, for example, the administration of an immunosuppressive to the transplant recipient. Immunosuppressives include, but are not limited to, corticosteroids (e.g., prednisolone or hydrocortisone), glucocorticoids, cytostatics, alkylating agents (e.g., nitrogen mustards (cyclophosphamide), nitrosoureas, platinum compounds, cyclophosphamide (Cytoxan)), antimetabolites (e.g., folic acid analogues, such as methotrexate, purine analogues, such as azathioprine and mercaptopurine, pyrimidine analogues, and protein synthesis inhibitors), cytotoxic antibiotics (e.g., dactinomycin, anthracyclines, mitomycin C, bleomycin, mithramycin), antibodies (e.g., anti-CD20, anti-IL-1, anti-IL-2Ralpha, anti-T-cell or anti-CD-3 monoclonals and polyclonals, such as Atgam, and Thymoglobuline), drugs acting on immunophilins, ciclosporin, tacrolimus, sirolimus, interferons, opioids, TNF-binding proteins, mycophenolate, fingolimod and myriocin. In some embodiments, anti-rejection therapy comprises blood transfer or marrow transplant. Therapies can also include therapies for treating systemic conditions, such as sepsis. The therapy for sepsis can include intravenous fluids, antibiotics, surgical drainage, early goal directed therapy (EGDT), vasopressors, steroids, activated protein C, drotrecogin alfa (activated), oxygen and appropriate support for organ dysfunction. This may include hemodialysis in kidney failure, mechanical ventilation in pulmonary dysfunction, transfusion of blood products, and drug and fluid therapy for circulatory failure. Ensuring adequate nutrition—preferably by enteral feeding, but if necessary by parenteral nutrition—can also be included particularly during prolonged illness. Other associate therapies can include insulin and medication to prevent deep vein thrombosis and gastric ulcers. Therapies for treating a recipient of a transplant can also include therapies for treating a bacterial, fungal and/or viral infection. Such therapies are known to those of ordinary skill in the art.

Similarly, the therapies can be therapies for treating cancer, a tumor or metastasis, such as an anti-cancer therapy. Such therapies include, but are not limited to, antitumor agents, such as docetaxel; corticosteroids, such as prednisone or hydrocortisone; immunostimulatory agents; immunomodulators; or some combination thereof. Antitumor agents include cytotoxic agents, chemotherapeutic agents and agents that act on tumor neovasculature. Cytotoxic agents include cytotoxic radionuclides, chemical toxins and protein toxins. The cytotoxic radionuclide or radiotherapeutic isotope can be an alpha-emitting or beta-emitting. Cytotoxic radionuclides can also emit Auger and low energy electrons. Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents include dolastatins (U.S. Pat. Nos. 6,034,065 and 6,239,104) and derivatives thereof. Toxins also include poisonous lectins, plant toxins such as ricin, abrin, modeccin, botulina and diphtheria toxins. Other chemotherapeutic agents are known to those skilled in the art. Examples of cancer chemotherapeutic agents include, but are not limited to, irinotecan (CPT-11); erlotinib; gefitinib (Iressa™); imatinib mesylate (Gleevec); oxalipatin; anthracyclins-idarubicin and daunorubicin; doxorubicin; alkylating agents such as melphalan and chlorambucil; cis-platinum, methotrexate, and alkaloids such as vindesine and vinblastine. In some embodiments, further or alternative cancer treatments are contemplated herein, such as radiation and/or surgery.

Administration of a treatment or therapy may be accomplished by any method known in the art (see, e.g., Harrison's Principle of Internal Medicine, McGraw Hill Inc.). Preferably, administration of a treatment or therapy occurs in a therapeutically effective amount. Administration may be local or systemic. Administration may be parenteral (e.g., intravenous, subcutaneous, or intradermal) or oral. Compositions for different routes of administration are well known in the art (see, e.g., Remington's Pharmaceutical Sciences by E. W. Martin).

As used herein, “a therapeutically effective amount” is an amount sufficient to provide a medically desirable result, such as treatment of transplant rejection, treatment of systemic disease, or treatment of cancer. The effective amount will vary with the particular condition being treated, the age and physical condition of the subject being treated, the severity of the condition, the duration of the treatment, the nature of any concurrent therapy, the specific route of administration and the like factors within the knowledge and expertise of the health practitioner. For administration to a subject such as a human, a dosage of from about 0.001, 0.01, 0.1, or 1 mg/kg up to 50, 100, 150, or 500 mg/kg or more can typically be employed. When administered, a treatment or therapy may be applied in pharmaceutically-acceptable amounts and in pharmaceutically-acceptable compositions. Such preparations may routinely contain salt, buffering agents, preservatives, compatible carriers, and optionally other therapeutic agents.

In some aspects, the disclosed methods for quantifying cell lysis and/or quantifying non-self cfDNA in a sample may be performed at one site, and treatment may be provided to the subject at a different site. Sample collection may occur at a separate site, or at the same site as one or more quantification methods are performed or the treatment is provided to the subject. For example, the sample may be obtained from the subject at a clinic and sent to a second site for quantification of cell lysis and/or non-self cfDNA in the sample. The amount of cell lysis and/or non-self cfDNA in the sample may be quantified at the second site and information regarding the same may be provided to a physician to guide administration of the appropriate therapy to the subject. Administration of the appropriate therapy may be provided at the clinic or at a separate third site.

In another aspect, compositions and kits comprising one or more primer pairs and/or probes as provided herein are provided. Other reagents for performing an assay, such as a PCR assay, may also be included in the composition or kit.

### EXAMPLES

### Example 1: Detection and Quantification of Cell Lysis

Experiments were performed to determine if Alu ratios (ALU 247/ALU 115) can detect white blood cell lysis when the white blood cells (buffy cells) of a sample are spiked into a plasma sample from a second individual with a low cell-free DNA concentration.

Whole blood was drawn from a first individual into an EDTA blood collection tube. The sample was spun at 1400 rpm for 10 minutes to separate the plasma and buffy coat. The buffy cells were carefully removed and resuspended in 1.0 mL of plasma from the first individual. The cells were counted (cells/μL) from a 500 μL aliquot. After quantification, the cells were diluted to a concentration of 100 cells/μL in PBS and then mixed into 2.0 mL of plasma from the second individual as described below.

Whole blood was drawn from the second individual into Streck BCT (blood collection tubes). The tubes were spun at 1400 rpm for 10 minutes, at which point the plasma was transferred to fresh 15 mL conical tubes. Approximately 1-1.5 mL of plasma was left in the BCT tubes to ensure that the buffy cells were not accidentally transferred during the process. The newly-transferred plasma was then centrifuged at 1400 rpm for another 10 minutes followed by a 10-minute spin at 15000 rpm. The plasma from all of the 15 mL conical tubes was then transferred and combined into a single 50 mL conical tube, where it was mixed and aliquoted (2 mL/tube). Different numbers of white blood cells from the first individual were then spiked into the samples, so that the tubes had 0, 250, 500, 1000, or 5000 cells per tube.

The plasma spiked with white blood cells were frozen overnight. These samples were then thawed at 37° C. and prepared for cfDNA extraction on a TECAN liquid handler. Three microliters were withdrawn from the eluate for RNaseP quantification, while 2 μL were used for the Alu assay.

For the Alu assay, samples were run in triplicate following a standard TaqMan® protocol using the following probe and primers:

Probe:
(SEQ ID NO: 1)
5′ CCAGCCTGGCCAACATGGTG 3′
Primers:
ALU 115
Forward:
(SEQ ID NO: 2)
5′-CCTGAGGTCAGGAGTTCGAG-3′
Reverse:
(SEQ ID NO: 3)
5′-CCCGAGTAGCTGGGATTACA-3′
ALU247
Forward:
(SEQ ID NO: 4)
5′-GTGGCTCACGCCTGTAATC-3′
Reverse:
(SEQ ID NO: 5)
5′-CAGGCTGGAGTGCAGTGG-3′

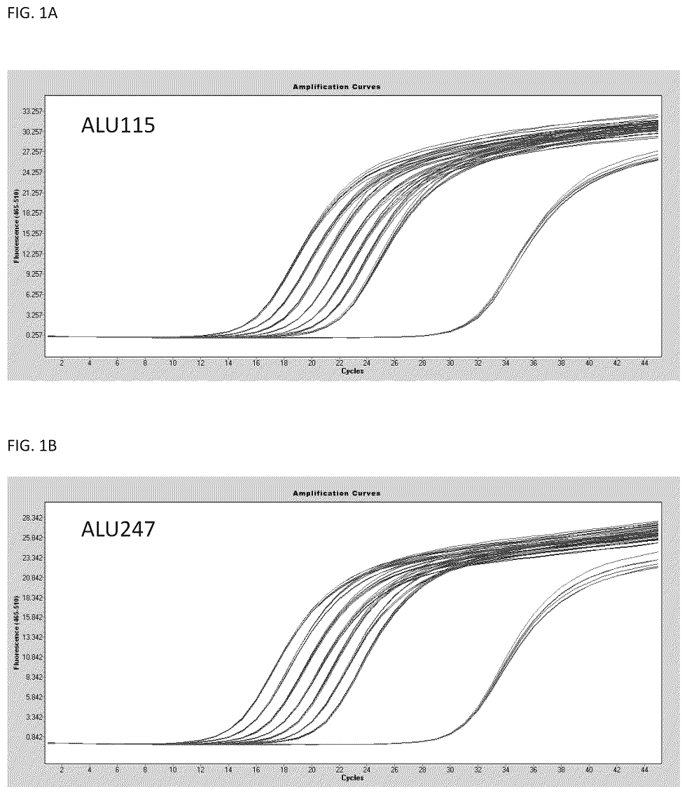

The results are shown FIG. 1A and FIG. 1B. Results are quantified in Table 1 below.

TABLE 1
ALU SYBR

| | | mean | | mean | |
|---|---|---|---|---|---|
| Sample ID | mean | normalized | mean | normalized | mean normalized |
| replicates | ALU247 [ng] | ALU247 [ng] | ALU115 [ng] | ALU115 [ng] | ALU247/115 ratio |
| 0 buffy cells_1 | 0.050 | 0.096 | 0.170 | 0.330 | 0.291 |
| 0 buffy cells_2 | 0.053 | 0.105 | 0.184 | 0.369 | 0.285 |
| 250 buffy cells_1 | 0.063 | 0.088 | 0.207 | 0.288 | 0.306 |
| 250 buffy cells_2 | 0.071 | 0.115 | 0.235 | 0.380 | 0.303 |
| 500 buffy cells_1 | 0.070 | 0.113 | 0.205 | 0.334 | 0.338 |
| 500 buffy cells_2 | 0.073 | 0.096 | 0.225 | 0.297 | 0.323 |
| 1000 buffy cells_1 | 0.049 | 0.079 | 0.133 | 0.214 | 0.369 |
| 1000 buffy cells_2 | 0.047 | 0.069 | 0.138 | 0.203 | 0.340 |
| 5000 buffy cells_1 | 0.164 | 0.204 | 0.248 | 0.309 | 0.660 |
| 5000 buffy cells_2 | 0.192 | 0.231 | 0.291 | 0.352 | 0.656 |

### Example 2—Cell Lysis Assay

The assay used a probe sequence set forth as: 5′ CCAGCCTGGCCAACATGGTG 3′ (SEQ ID NO: 1) and TaqMan™ chemistry. The probe has 6FAM on the 5′ end and is an MGB probe with a non-fluorescent quencher at the 3′ end. The resulting assay was quite sensitive, with 20 pg easily within the linear quantifiable range. The test can, therefore, be used to assess DNA fragment lengths at even smaller amounts than previously described in the literature.

### Cell Lysis Experiment 1

The experiment was designed to determine whether Alu ratios can detect white cell lysis with 0, 250, 500, 1000 and 5000 cells spiked (individual 1) into plasma (individual 2) with low ng/ml cfDNA concentration.

The procedure was conducted as follows:

Individual 1 was selected as a source of buffy coat.

Individual 2 was selected as a source of plasma (˜16 ng/ml total cfDNA). The sample was collected in EDTA and left out at room temperature for days in order to allow cells to lyse.

A whole blood sample was drawn from individual 1 into a purple capped EDTA blood collection tube. The tube was spun to separate plasma and buffy coat while leaving RBCs (1400 rpm for 10 minutes). Buffy cells were resuspended in 1.0 ml of plasma from Individual 1. Following resuspension, cells were counted using Cell Dyne (expressed as #cells/μl) using 500 ul aliquots. Once the cells were counted, the buffy cell samples were diluted into PBS and then cells were mixed into 1.0 ml of plasma from individual 2 to generate samples containing 5000 cells, 1000 cells and 500 cells, 250 cells and no cells (from individual 1), respectively. Tubes were then frozen overnight.

The following day, cfDNA was extracted and RNaseP was quantified to calculate the total cfDNA (ng/ml plasma). Alu 247/115 ratio were then measured. Levels of cf-DNA were determined to be too high to mimic normal cell-free DNA from healthy individuals based upon the Alu 247/115 ratio results from this experiment.

### Cell Lysis Experiment 2

Purpose: To see if Alu ratios can detect white cell lysis with 0, 250, 500, 1000, and 5000 cells spiked (individual 1) into plasma (individual 2) with low ng/ml cfDNA concentration.

The procedure was conducted as follows:

Individual 1 was selected as a source of buffy coat.

Individual 2 was selected as a source of fresh plasma. To generate plasma, a whole blood sample was also from individual 2 into Streck BCT blood collection tubes. BCT tubes were centrifuged at 1400 rpm for 10 minutes and plasma was transferred to a fresh 15 ml conical tube (left about 1-1.5 ml plasma from buffy coat layer). Tubes containing the plasma were centrifuged for another 10 minutes at 1400 rpm, and plasma was transferred to fresh 15 ml conical tubes (left about 0.5 ml plasma from the bottom) and spun 10 minutes at 15000 rpm. All plasma was combined into a 50 ml conical tube, mixed, and then 2 ml were aliquoted to 50 ml conical tubes (a total 10 aliquots). Varying numbers of white blood cells were spiked into the 50 ml conical tubes.

A whole blood sample was collected from individual 1 into a purple capped EDTA blood collection tube. The tube was spun to separate plasma and buffy coat, while leaving RBCs (1400 rpm for 10 minutes). Buffy cells were resuspended in 1.0 ml of plasma from individual 1. Following resuspension, cells were counted using Cell Dyne (expressed as #cells/μl) using 500 ul aliquots. Once the cells were counted, the buffy cell samples were diluted into PBS and then cells were mixed into 2.0 ml of plasma from individual 2 (obtained as described above) to generate samples containing 5000 cells, 1000 cells and 500 cells, 250 cells and no cells (from individual 1), respectively. Tubes were then frozen overnight.

The following day, tubes were thawed at 37° C. and the spiked samples were transferred to a 50 ml conical tube for TECAN cf-DNA extraction. RNaseP was quantified and ALU 247/115 ratios were measured.

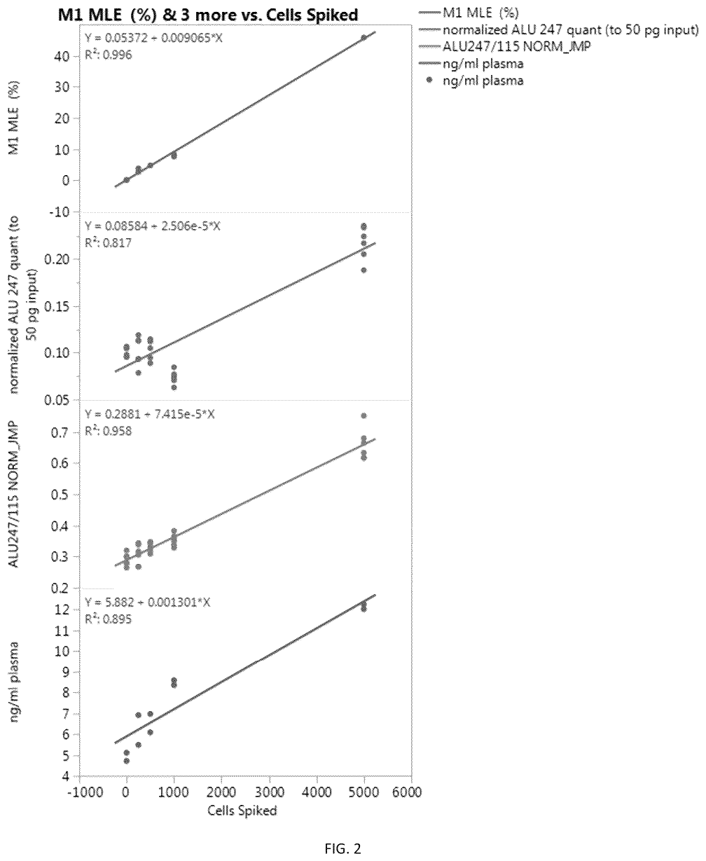

Results are shown in FIG. 2. Alu 247/115 ratios increased linearly and were sensitive across all cell amounts lysed into the plasma.

### Example 3—ALU Cell Lysis Assay

ALU Assay: The main source of cell fee DNA (cfDNA) in healthy people is from normal apoptotic processes. These processes enzymatically cleave the DNA into short fragments of ˜185-200 base pairs (Umetani, N., Giuliano, A. E., Hiramatsu, S. H., Amersi, F., Nakagawa, T., Martino, S., Hoon, D. S. B., 2006. Prediction of Breast Tumor Progression by Integrity of Free Circulating DNA in Serum. Journal of Clinical Oncology 24, 4270-4276). When cells undergo non-apoptotic lysis, DNA fragments sizes vary in length and are generally longer than fragments created by apoptotic processes. This non-apoptotic lysis can occur for many reasons. For example, non-apoptotic lysis can occur when non-stabilized lymphocytes are stored at room temperature for extended periods of time or are exposed to agitation. Additionally, if any of the sample preparation methods allowed white blood cells to be included in the sample just prior to DNA extraction, those WBCs could have artificially reduced the reported Donor Fraction (DF).

ALU 115 bp amplification detects both shorter fragments from apoptosis as well as longer fragments derived from non-apoptotic lysis. Only longer fragments derived from non-apoptotic lysis are detected by ALU 247 bp amplification. The ratio of longer ALU 247 fragments to ALU 115 fragments has been found to increase as lysis increases, providing a useful tool to measure cell lysis in samples, particularly those for non-self cfDNA analysis.

The ALU data shown in this example represents the mean of three wells in a 384 well plate. ALU 115 and ALU 247 CVs for the three points are captured. All ALU data was verified via an R script.

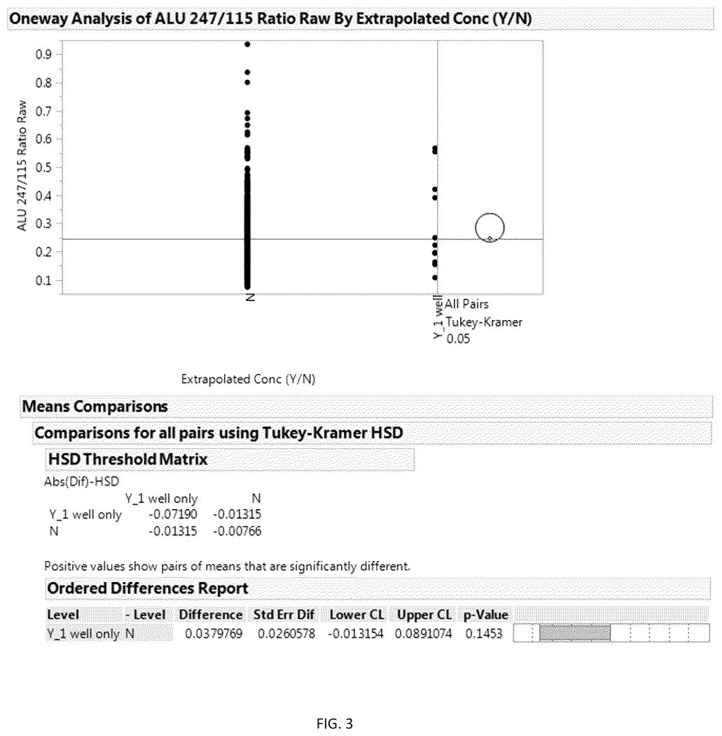

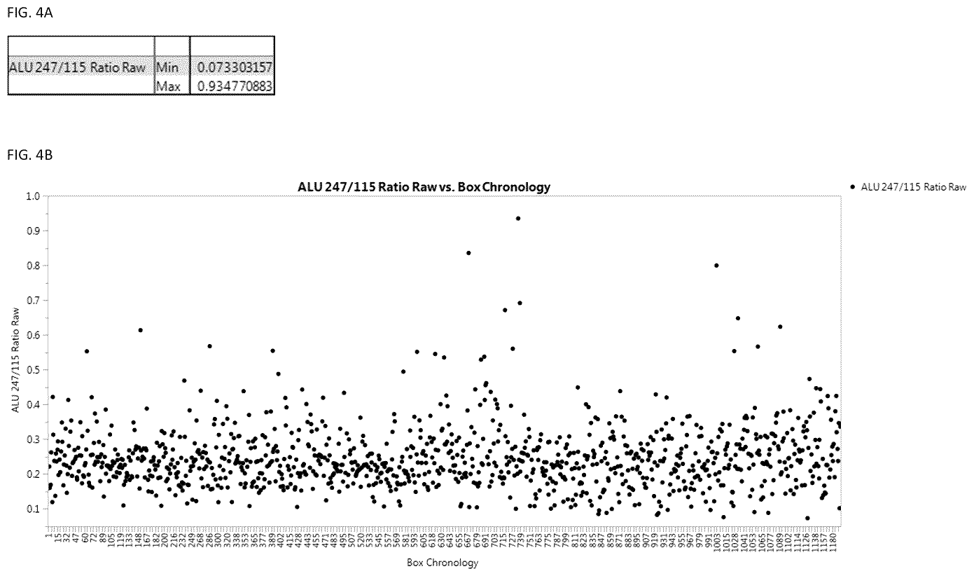

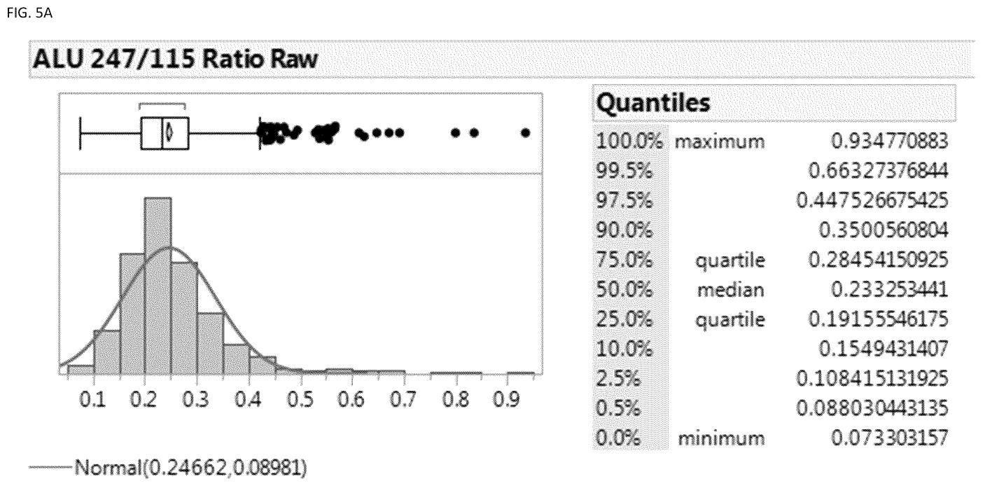

1075 samples were collected and used for the ALU analysis. There were 7 samples that measured outside of either ALU 115 or ALU 247 standard curves. Results for these samples are not included in the ALU analysis. The remaining 1068 samples were used to generate the data shown in FIG. 3. This 1068 samples includes 12 samples that yielded 1 well of three that fell outside either the ALU 115 or ALU2 47 standard curve. Results for these samples are included in the ALU analysis as their ALU ratio falls within the distribution of the non-extrapolated sample ALU ratios (FIG. 3, p=0.1453).

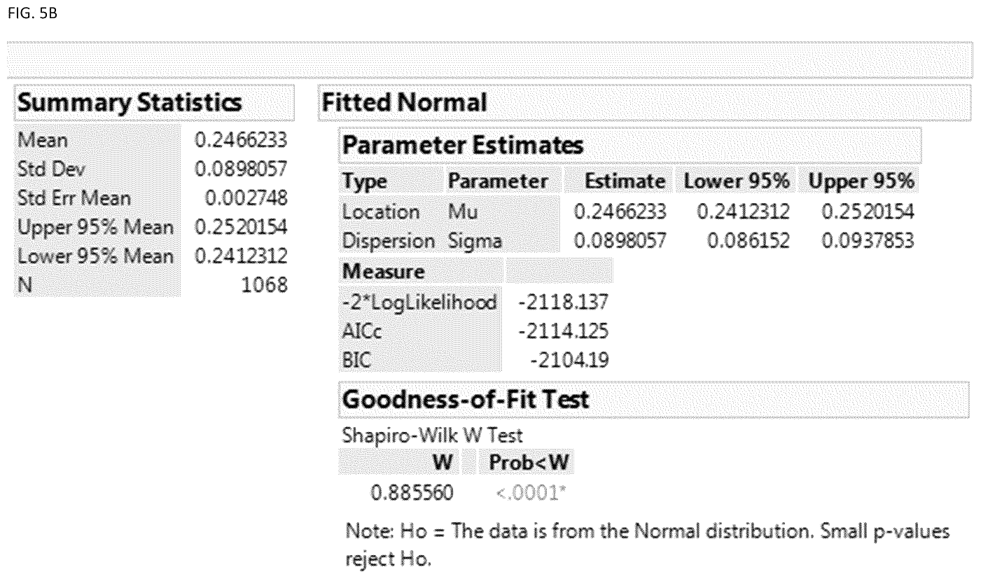

ALU Ratio Data: ALU 247/115 ratio data for the 1068 samples included in the analysis ranges from 0.07-0.93 (FIG. 4A). Box chronology indicates the order the samples were run, from lowest (Day 1) to highest (Day 5) (FIG. 4B). The ALU247/115 ratio and statistics are shown in FIG. 5A-5B. The data is not normal. 39 points are considered outliers. The maximum ALU ratio excluding outliers is 0.42.

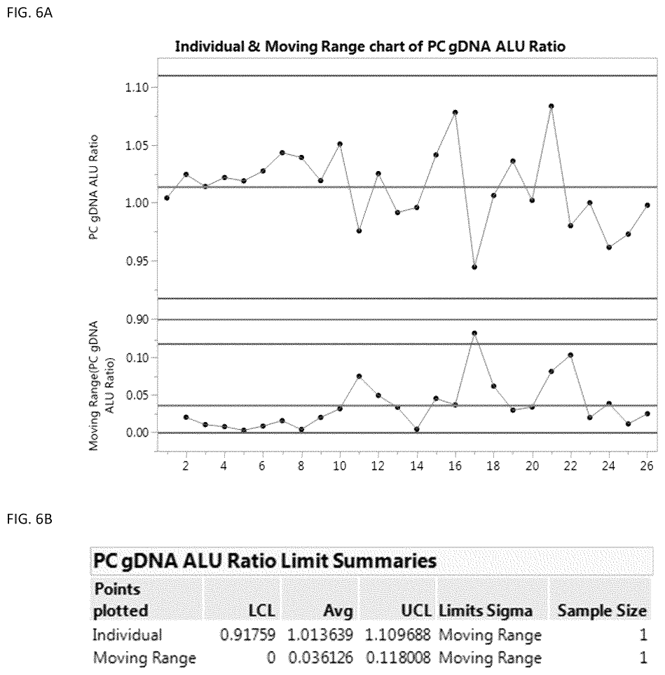

Positive Control: gDNA ALU Ratio Across all 26 plates: The ALU ratio of the highest gDNA standard (500 pg) for each ALU assay was calculated and all 26 points were analyzed via JMP control chart (FIG. 6A-6B). It was expected that this ALU ratio would be ˜1, as there should be equal long and short fragments in gDNA. This range encompasses the variability from different days, runs, operators, and instruments. Variation is expected within the Lower Control Limit (LCL) and Upper Control Limit (UCL) when the process is in statistical control. The LCL from the 26 ALU runs is 0.92. The UCL from the 26 ALU runs is 1.11.

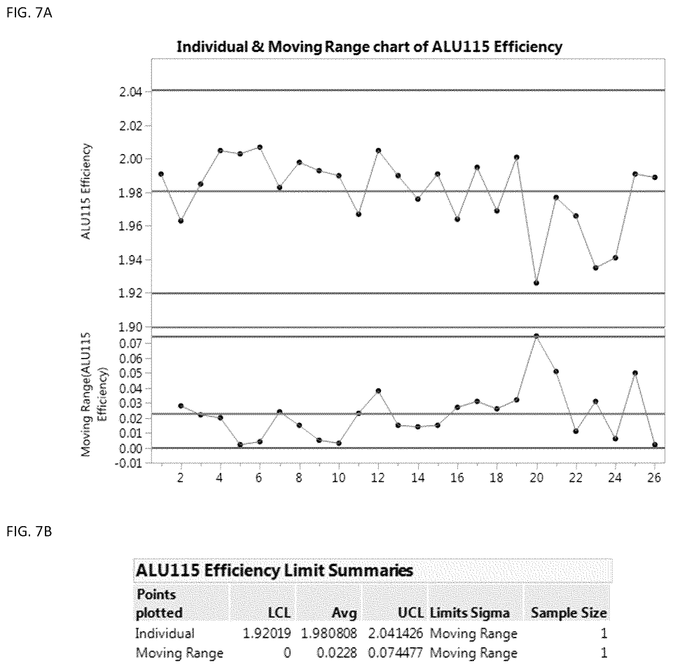

ALU 115 PCR Efficiency Across all 26 plates: The efficiency of the ALU 115 PCR was captured and all 26 runs were analyzed via JMP control chart (FIG. 7A-7B). This range encompasses the variability from different days, runs, operators, and instruments. Variation is expected within the Lower Control Limit (LCL) and Upper Control Limit (UCL) when the process is in statistical control. The LCL from the 26 ALU runs is 1.92. The UCL from the 26 ALU runs is 2.04.

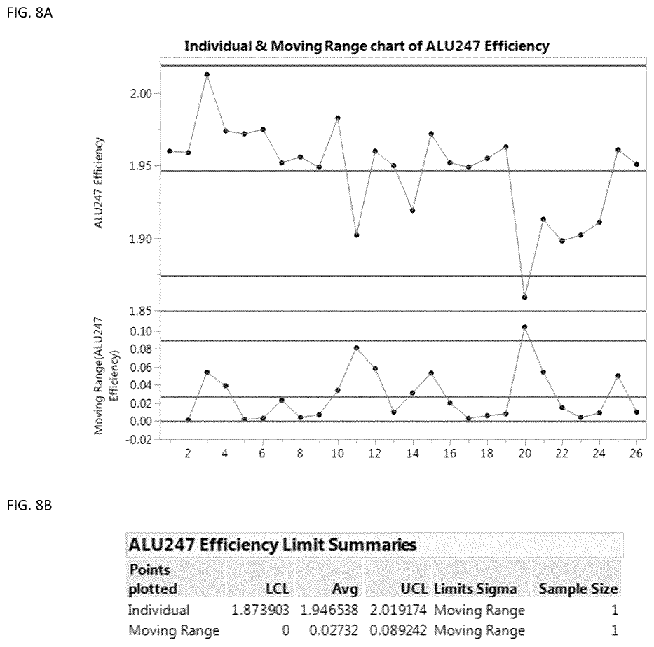

ALU 247 PCR Efficiency Across all 26 plates: The efficiency of the ALU 247 PCR was captured and all 26 runs were analyzed via JMP control chart (FIG. 8A-8B). This range encompasses the variability from different days, runs, operators, and instruments. Variation is expected within the Lower Control Limit (LCL) and Upper Control Limit (UCL) when the process is in statistical control. The LCL from the 26 ALU runs is 1.87. The UCL from the 26 ALU runs is 2.02.

Sample Preparation Analysis—Spin Protocol: ALU ratio and total DNA via RNase P was analyzed for samples and correlated to how the sample plasma was prepared. 1054 samples were included in this analysis: after the data was correlated with quality metrics, 16 samples were removed. Of these, 136 were removed as missing or falling outside the standard curve measurement, leaving 1054 as the data set the following Spin Protocol Analysis is based upon.

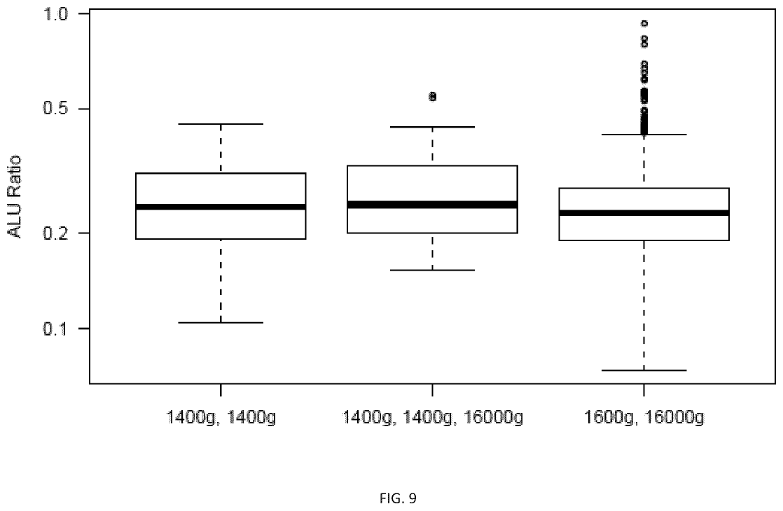

ALU Ratio—Spinning samples at 1600×g first, followed by spinning at 16000×g yields more samples with a higher ALU ratio when compared to two spins at 1400×g and two spins at 1400×g followed by a 16000×g spin (FIG. 9). It is thought that two spins at 1400×g removes all WBCs from the sample, and therefore there are no cells to lyse and contribute to the longer ALU 247 fragment. When plasma is spun only once at 1600×g, there are still WBCs in the plasma that are lysed upon the extreme 16000×g spin.

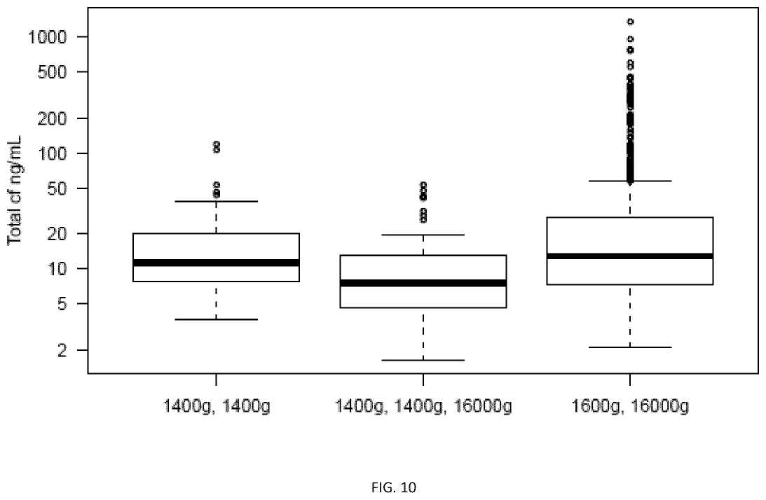

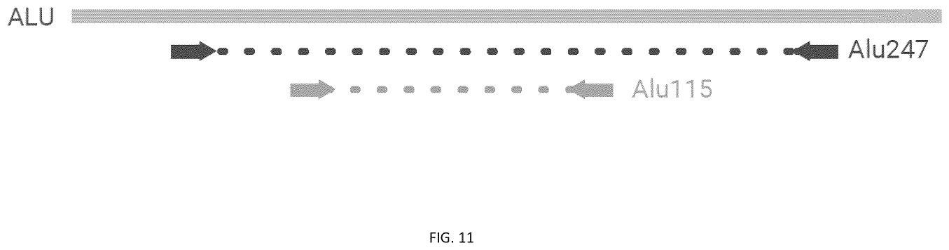

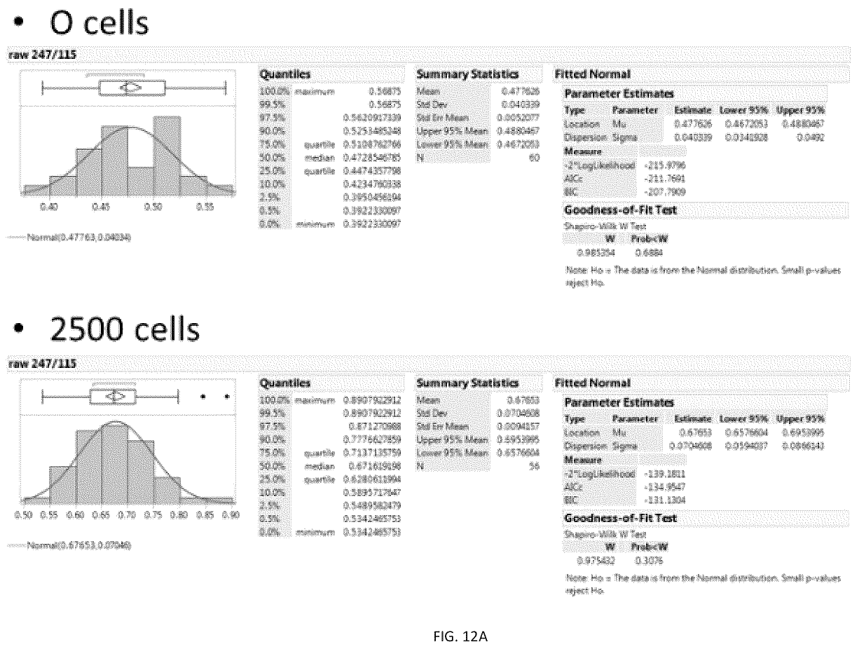

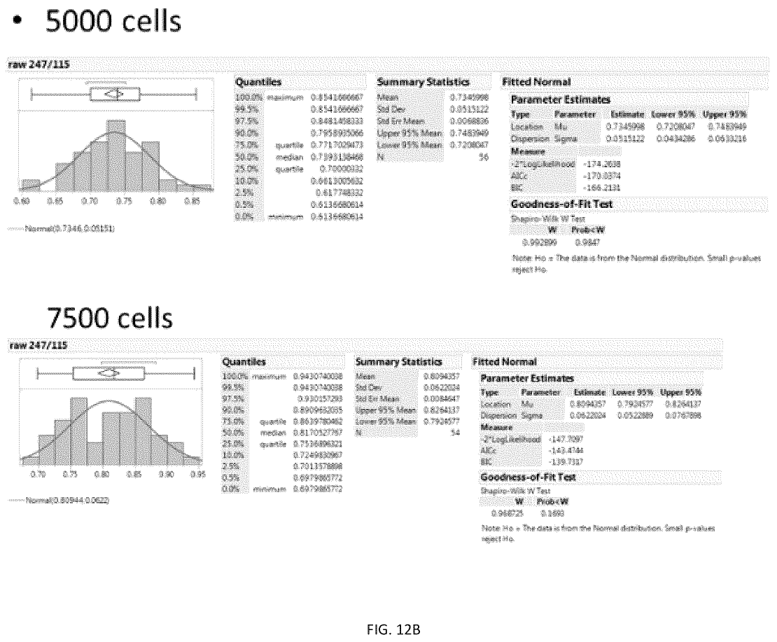

Total DNA Detected via RNase P— Spinning samples at 1600×g first, followed by spinning at 16000×g yielded more samples with higher total DNA detected when compared to two spins at 1400×g and two spins at 1400×g followed by a 16000×g spin (FIG. 10).

Measuring the ALU ratio for the samples described in the report above yields important information regarding the ALU ratios. Control charting the positive gDNA control ALU ratio, as well as the ALU 115 and ALU 247 efficiencies yields a good understanding of the variance when running ALU on samples.

### Example 4—ALU Characterization

Cell lysis can interfere with the measurement of non-self cfDNA. ALU repeats are the most abundant sequences in the human genome (copy number of ˜1.4 million per genome, accounting for more than 10% of the genome). The main source of cfDNA in healthy people is apoptosis (part of normal & controlled growth). DNA released from apoptosis is uniformly truncated to 185 bp-200 bp fragments. CfDNA released from tumor or lysed cells varies in length, containing more longer fragments. The ALU assay described herein measures long (247 bp) and short (115 bp) ALU repeats (FIG. 11).

Varying levels of cells (0 cells, 2500 cells, 5000 cells, and 7500 cells) were spiked into plasma. The cells were lysed, cf-DNA samples were extracted, and an Alu assay was performed. ALU247/115 ratios and calculated statistics for each cell amount as shown in FIGS. 12A-12B.

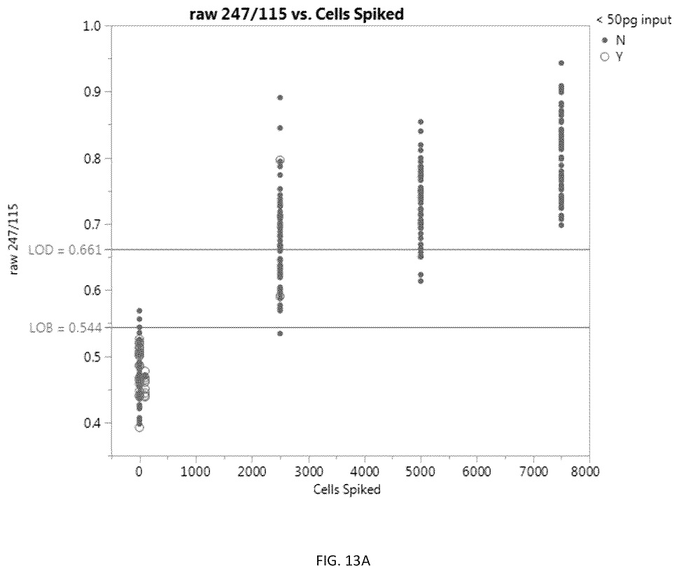

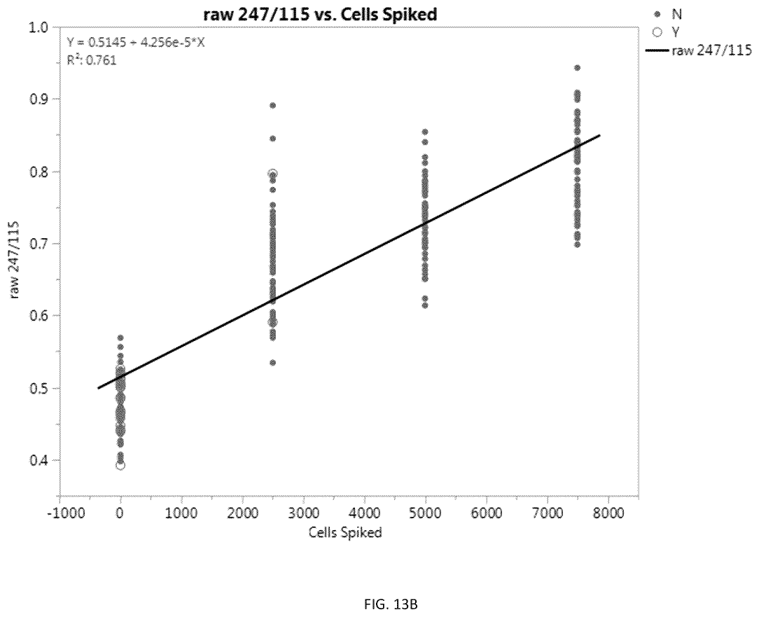

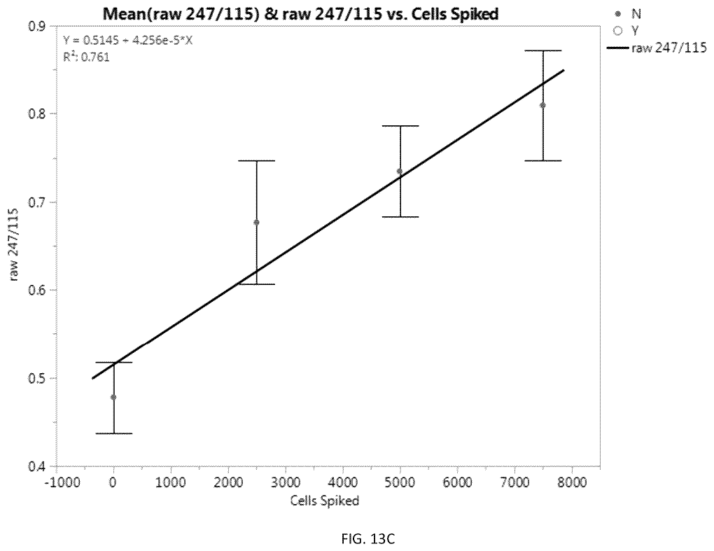

As the number of spiked cells in the sample increased, the ratio of ALU247/115 also increased. A clear separation between 0 cells spiked and >2500 cells spiked was observed (FIG. 13A, 13B, 13C; LOB=0.544, LOD=0.661.)

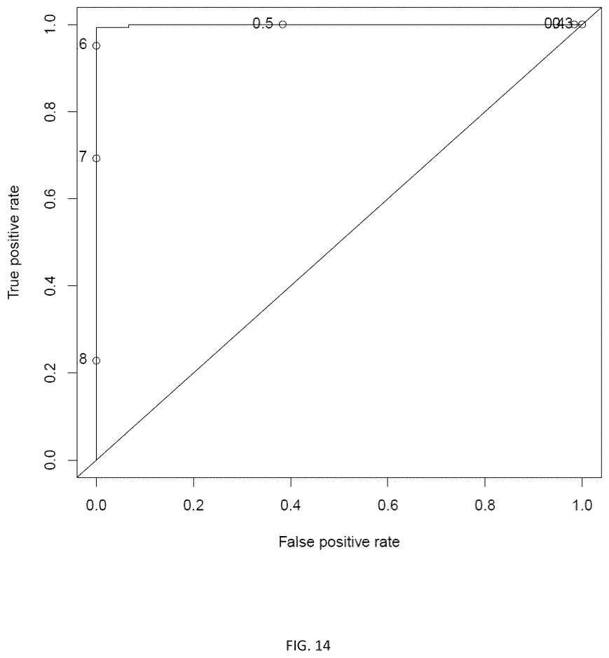

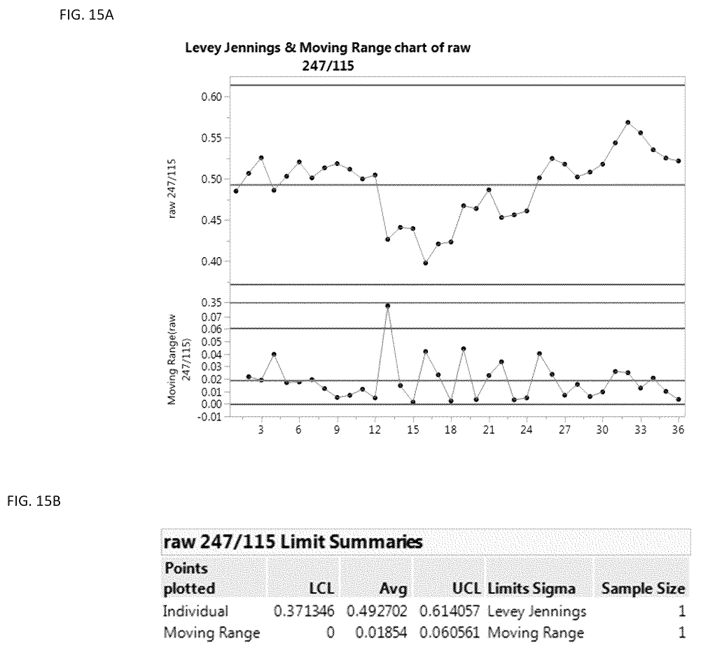

The ROC curve for the Ratio vs Cells Spiked was calculated. Setting a cutoff at 0.57 or 0.6 can differentiate ≥2500 cells lysed from 0 cells lysed with ˜95% sensitivity and 100% specificity. The true positive rate vs. false positive rate is shown in FIG. 14. Variability plots are shown in FIG. 15A. Over 3 days, the variability of un-spiked plasma extends up to an ALU ratio of 0.614057 (FIG. 15B). To differentiate from un-spiked plasma, spiked plasma should be above this ALU ratio for this example.

### ALU Characterization and Threshold

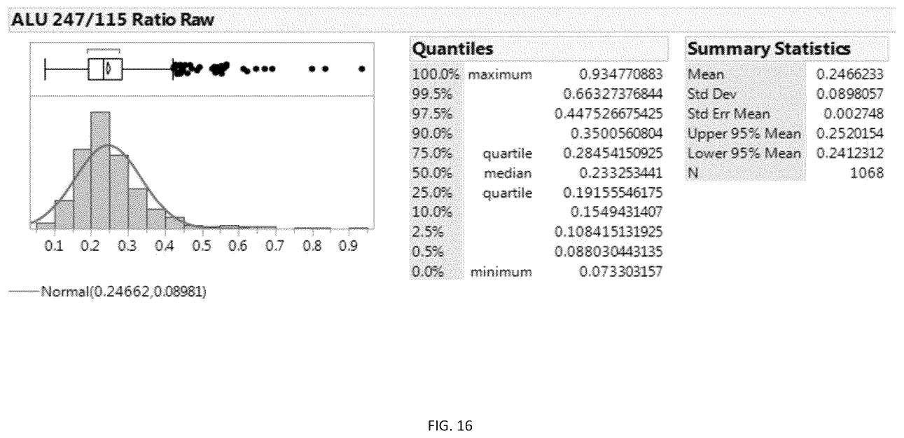

DNA contributed from about 2500 cells can be detected with an Alu assay ratio value versus 0 cells spiked. Thus, in this example an Alu ratio of about 0.49 corresponds to the number of lysed cells (FIG. 16). An Alu ratio of greater than 0.42 was seen to be outside of a normal distribution of samples in this example. The Alu ratio may be variable across subjects.

### Different Long Fragment and Short Fragment Primers

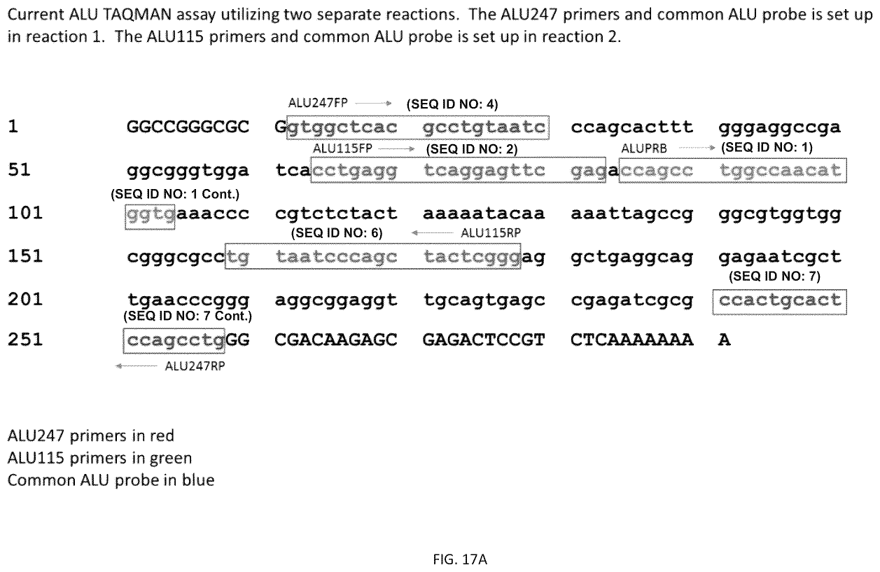

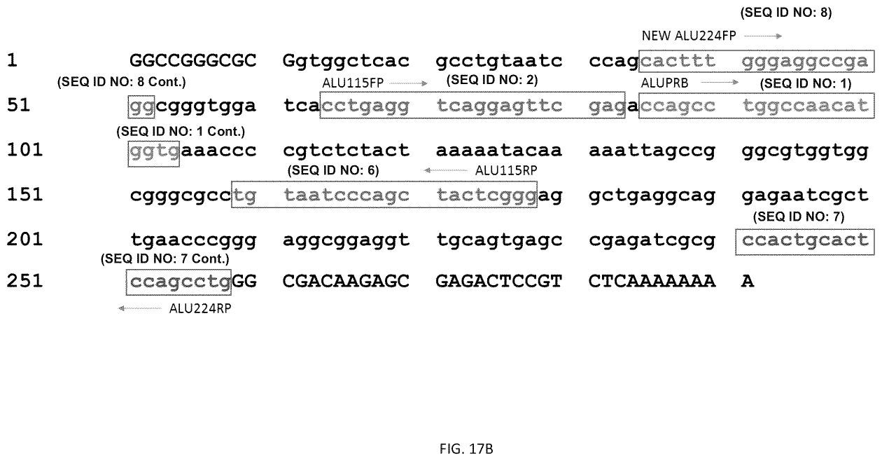

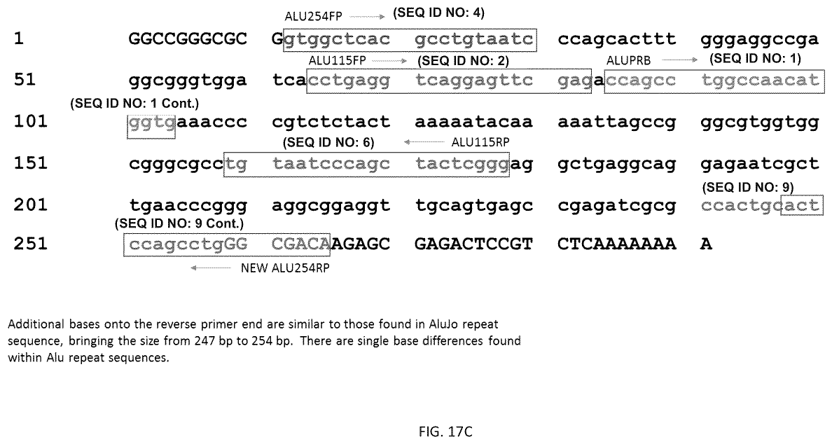

While the above examples were performed with primers for a long Alu fragment of 247 bps and short Alu fragment of 115 bps, the methods provided here can be performed with other primers to amplify fragments of these sizes or with primers that can amplify long and short Alu fragments of different lengths. Potential primers are shown in FIG. 17A-17C.

### Example 5—SYBR Versus TAQMAN

The methods provided herein can be performed with various RT-qPCR techniques including those that comprise SYBR or TAQMAN chemistries. In some embodiments, however, TAQMAN may be preferred as it was found to be more sensitive and quantitative in some examples. Thus, in any one of the methods provided herein the RT-qPCR is performed with TAQMAN chemistries.

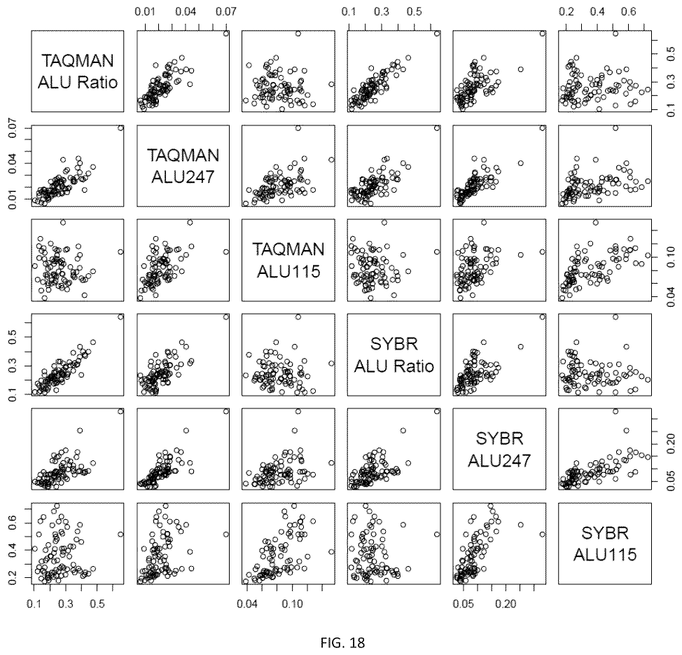

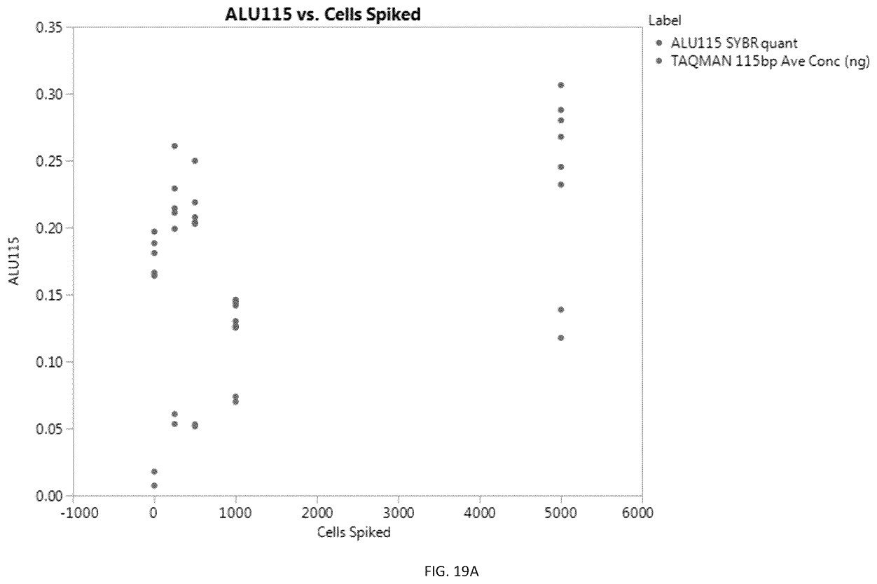

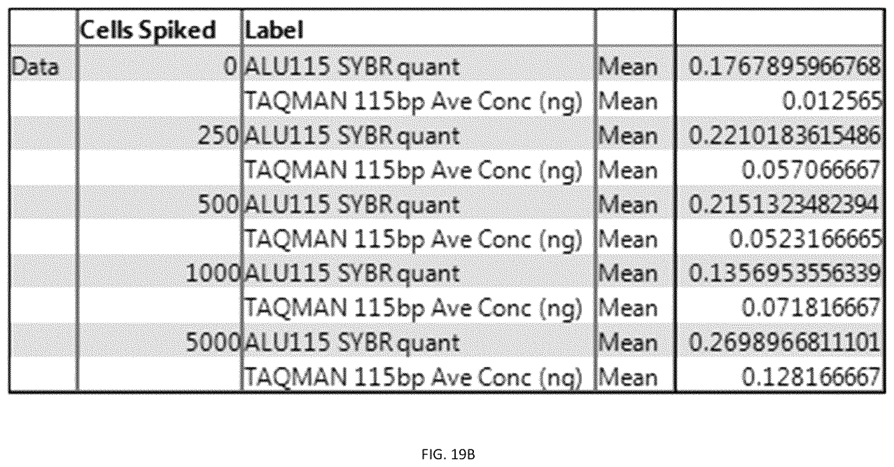

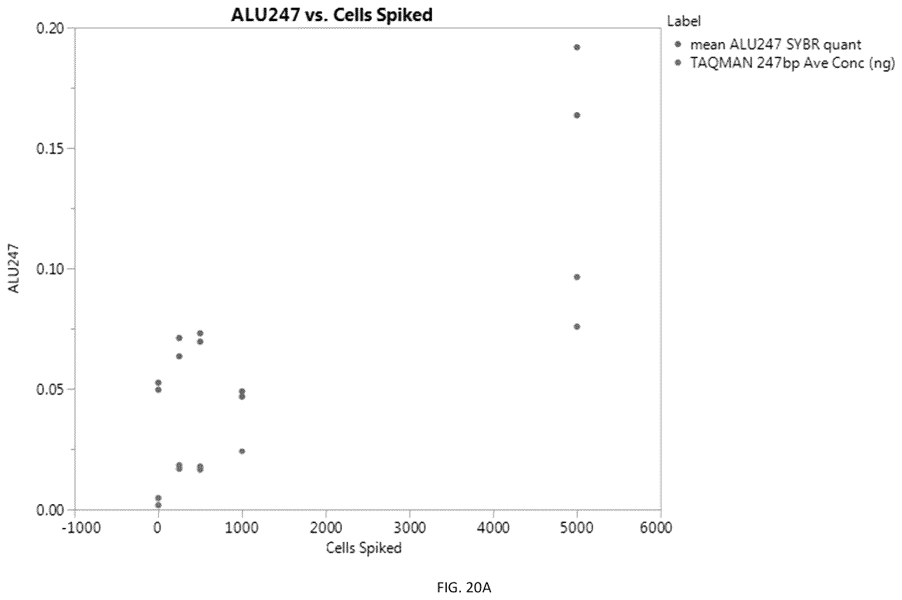

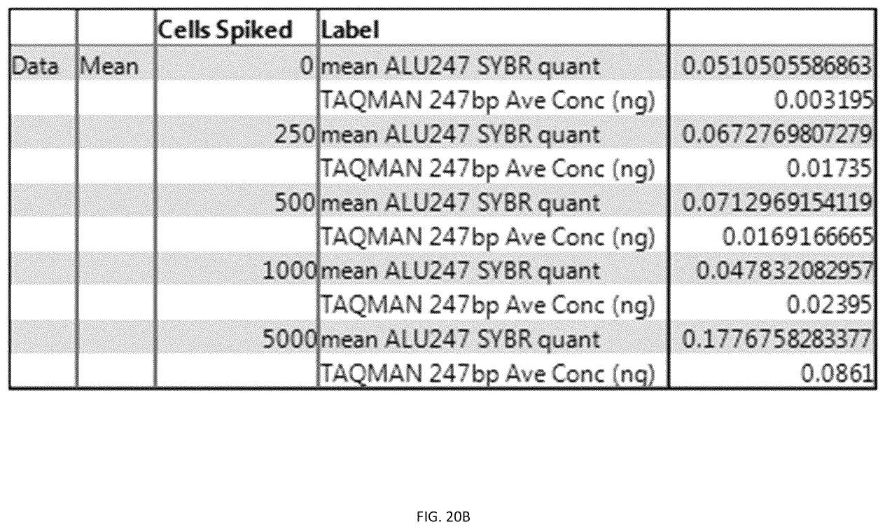

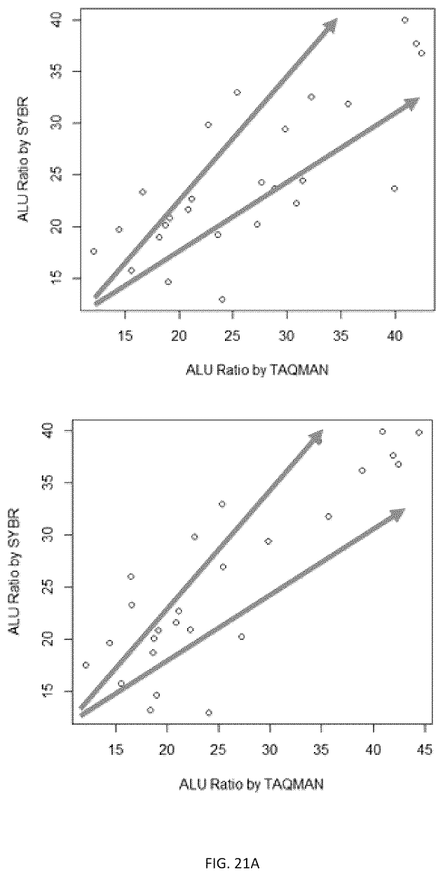

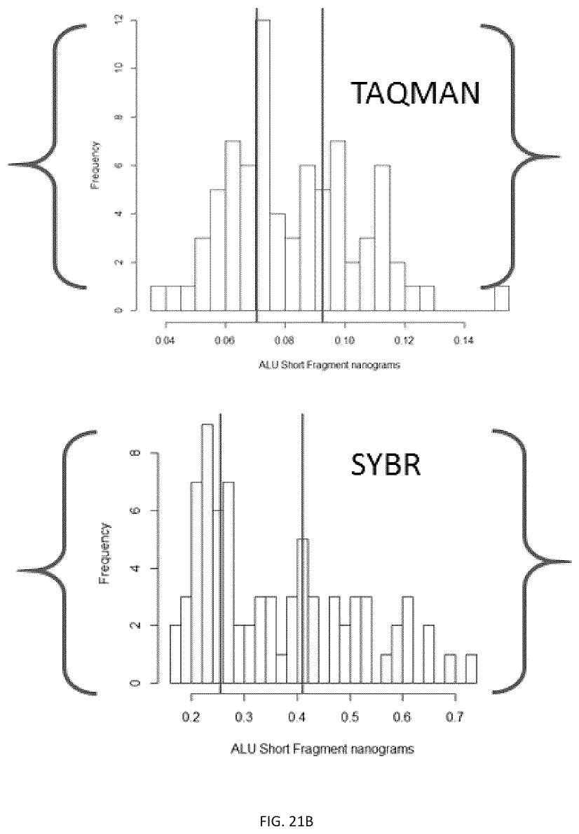

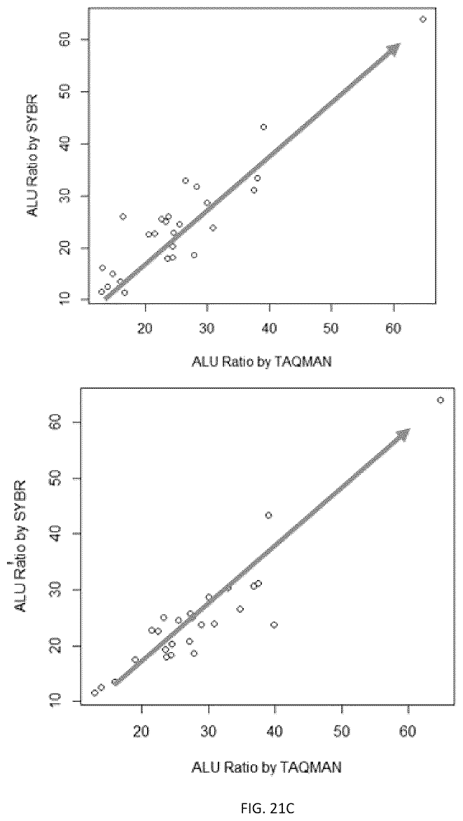

For example, a collection of 77 human patient samples spiked with varying amounts of cells were run both SYBR and RT-qPCR. The ALU Ratios were measured by both. (FIG. 18-22). ALU115 is shown in FIG. 19A-B. ALU247 is shown in FIG. 20A-B. The samples with lower total input DNA as measured by the short fragment (first tertile TAQMAN ALU 115 grams) had a lower correlation (76% vs 92%) between SYBR and TAQMAN ALU Ratios than the samples with higher total input DNA as measured by the short fragment (third tertile TAQMAN ALU 115 grams) Samples in the lower third of overall DNA quantity by TAQMAN correlate by 75.6% and by SYBR 85.6% (FIG. 21A). Samples in the upper third of overall DNA quantity by either technology correlate by 92% (FIG. 21B). A greater difference in correlations indicates that the TAQMAN assay has more precision in the lower input samples than the SYBR assay (FIG. 21C). This indicates that this assay was more reliable with lower DNA inputs on the TAQMAN chemistry.

Across all lysis levels, the SYBR assay's two ALU measurements (grams 115 and grams 247) were correlated more strongly (67% vs 52%) than the TAQMAN assay's two ALU measurements (grams 115 and grams 247). Therefore the dual assay in this example contained more mathematical information when using TAQMAN.

In addition, using SYBR, the difference between low and high DNA input samples was less stark than when samples were selected by TAQMAN in this Example. Thus, the certainty of the definition of low vs high was better in TAQMAN for this analysis.

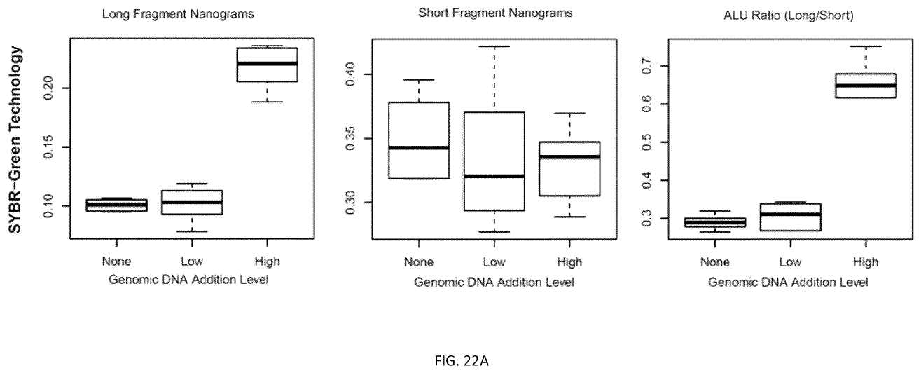

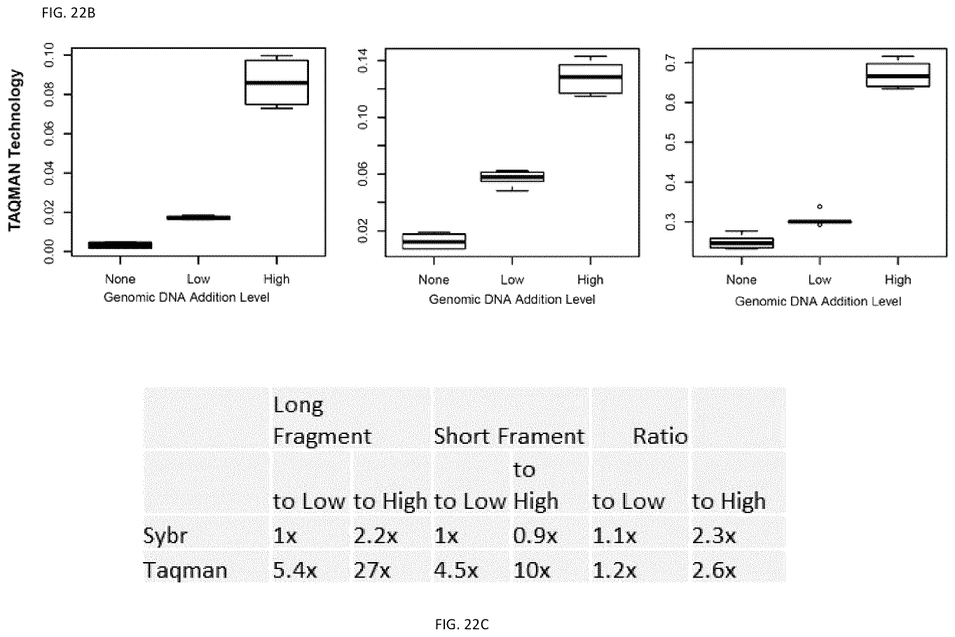

ALU assays comparing SYBR vs. TAQMAN in contrived samples (cell lysis Experiment 2) are shown in FIG. 22A-C. SYBR cannot distinguish the low level of DNA addition from background, whereas TAQMAN short and long fragment assays have twice the sensitivity (fold-change) to DNA additions (FIG. 22A-22C).

### Example 6—Multiplexing and Smaller Sample Size

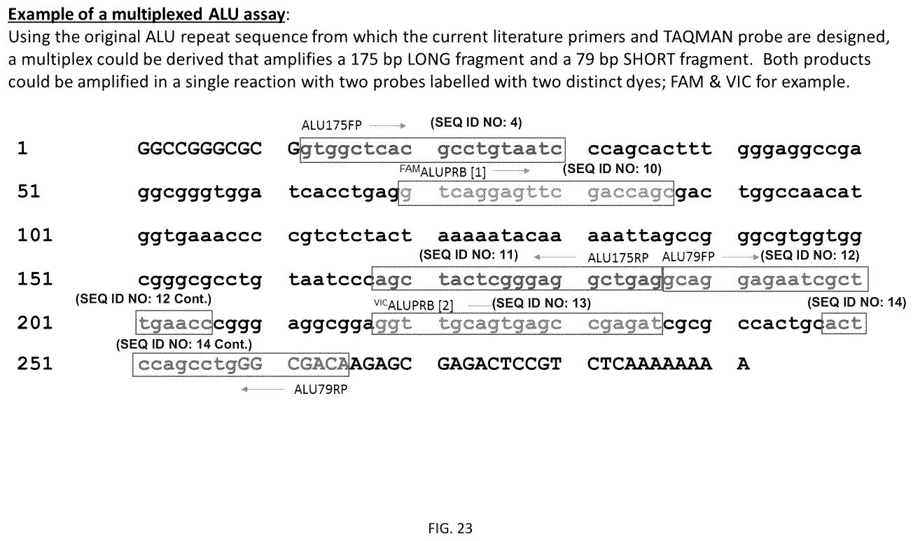

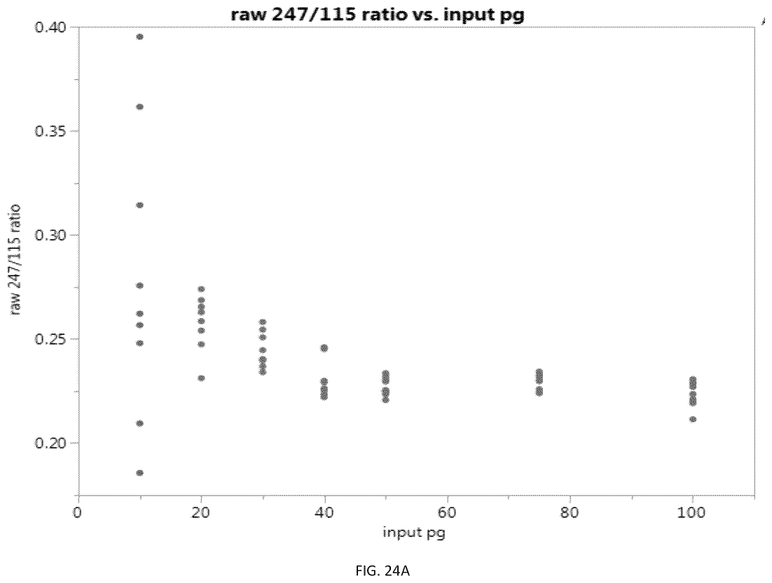

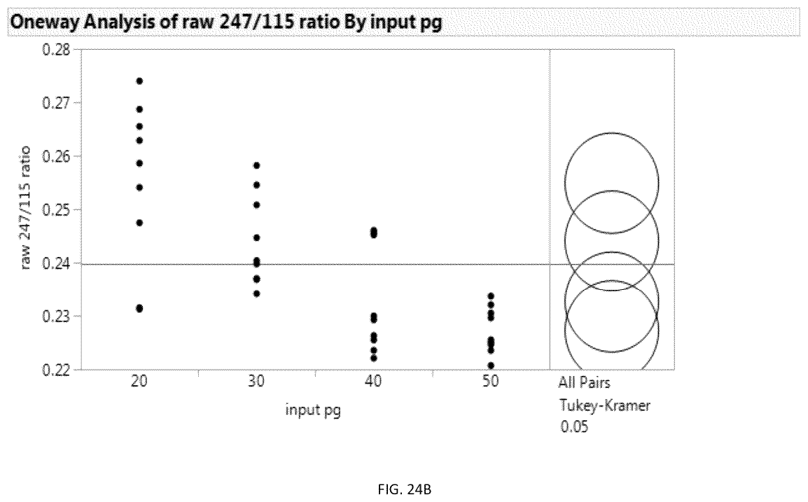

The methods provided herein offer significant sensitivity and accuracy even with very small sample sizes. Nevertheless, by the use of an additional probe for a region that does not overlap the short fragment, even smaller sample sizes can be analyzed. Thus, in any one of the methods provided herein the long and short fragment may be amplified in the same reaction. In addition, any one of the methods provided herein can comprise the use of an additional probe (e.g., for a region that does not overlap the short fragment). Examples of ALU primers and probes that may be used are shown in FIG. 23.

### Example 7—Variation in ALU Ratio Based Upon Amount of Input DNA

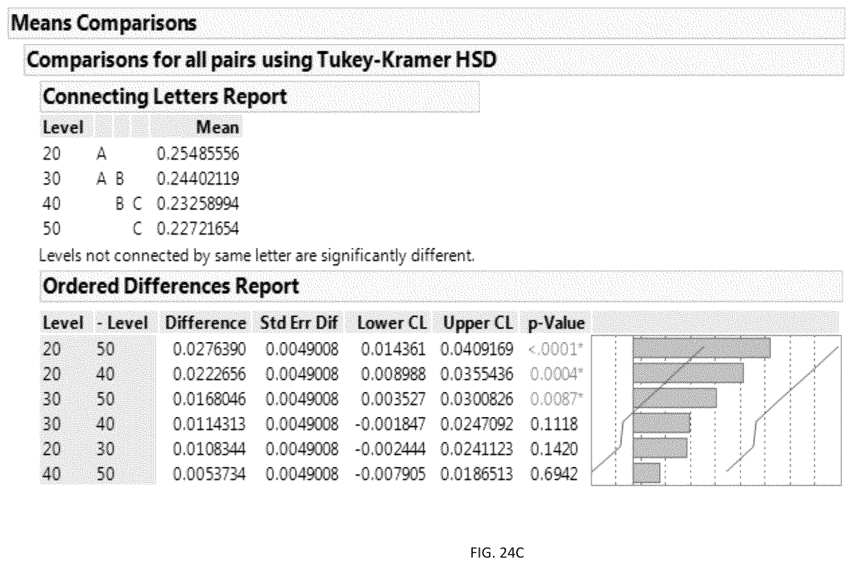

Experiments were conducted to test the effect of input DNA on the ALU247/115 ratio. Samples containing 10, 20, 30, 40, 50, 75, and 100 pg of DNA were tested in the ALU assay. Results are shown in FIG. 24A. One-way analysis of raw 247/115 ratio are shown in FIG. 24B. Results are quantified in FIG. 24C.

### Example 8—Quality Control to Detect Leukocyte Contamination/Lysis in Patient Samples

Reference Materials: The validation studies reported here were dependent upon large volumes of input plasma to test all variables in an appropriate number of replicates while also providing the appropriate range of cfDNA concentration and cfDNA DF. Accordingly, contrived reference materials consisting of specified combinations of human plasma samples, human cfDNA and genomic DNA isolates, and sheared human genomic DNA preparations were developed and manufactured at TAI Diagnostics to support validation study needs, including provision of controls. Unless otherwise stated, all plasma samples were isolated from whole blood sourced from a commercial vendor. Plasma was separated from whole blood by centrifuging at 1400×g for 10 minutes, removed and centrifuged a second time at 1400×g for 10 minutes, followed by a third centrifugation at 15,000×g for 10 minutes. Aliquots of the plasma and the buffy coats were frozen at −80° C. until needed. For use in validation studies of the DNA fragmentation test, plasma was spiked with short fragments of DNA obtained by Covaris ME220 focused ultrasonication (“shearing”) of genomic DNA from the paired cellular component (buffy coat) to a size distribution primarily in the range of 130-180 bp, approximating that of cfDNA. Resultant fragment lengths were evaluated on an Agilent Bioanalyzer (Santa Clara, CA) with a high sensitivity DNA chip to confirm production of the targeted range as determined by base pair size of maximum fluorescence values (below).

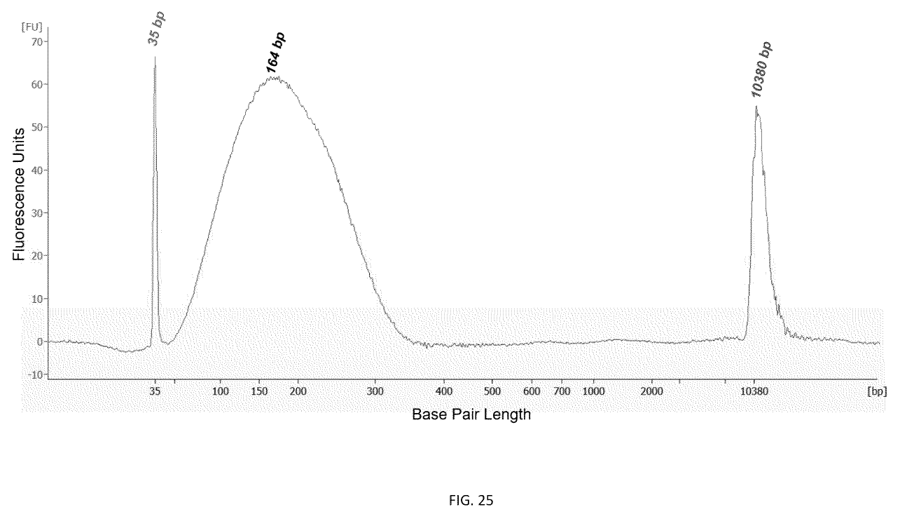

Electropherogram image of sheared gDNA, simulating cfDNA. An Agilent 2100 Bioanalyzer instrument and high sensitivity DNA Kit were used to demonstrate the 164 bp peak corresponding to the median distribution of gDNA. sheared by ultrasonication to the size range of cfDNA of apoptotic origin. FU, fluorescence units; bp, base pairs. Peaks at 35 and 10380 bp represent lower and upper internal kit standards (FIG. 25).

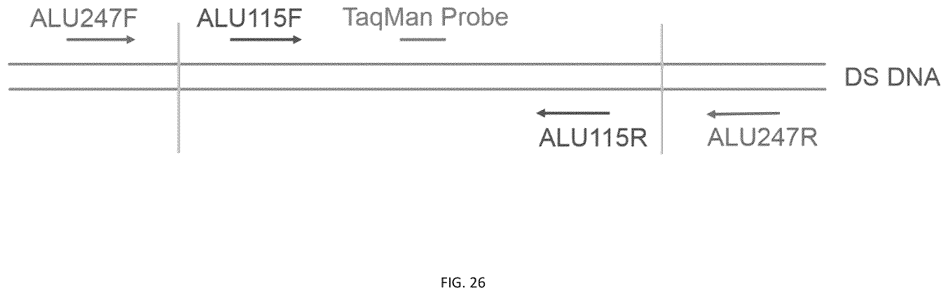

The ALU115 primer pair utilized produces amplification product from Alu fragments of almost all lengths, including the short fragments of a modal size of about 166 bp (140-200 bp) characteristically derived from cellular apoptosis, as well as all longer ALU fragments, essentially the entire cfDNA complement. In contrast, only those longer fragments derived from non-apoptotic cellular death mechanisms, such as those occurring from ex vivo lysis of leukocytes during whole blood sample processing, are detected by amplification of a 247 bp fragment of the Alu sequence (FIG. 26). The ratio (here referred to as “Alu Ratio”) of product from the ALU247 amplification to product from the ALU115 amplification increases as contribution by post-collection leukocyte lysis to the cfDNA pool increases. Because the annealing site of ALU-115 is nested within the ALU-247 annealing site, the qPCR ratio (DNA integrity) would theoretically be 1.0 when template DNA is not fragmented and 0.0 when all template DNA is truncated into fragments smaller than 247 bp. Because the ALU-115 primers can amplify most fractions of circulating DNA, the ALU-qPCR result obtained with ALU-115 primers in plasma samples effectively represents the absolute amount of cfDNA. The Alu ratio (247 bp:115 bp) provides a useful tool to detect levels of leukocyte lysis as the result of improper whole blood sample handling, which might produce a false negative result for increased probability of rejection in samples evaluated for DF.

Alu 115 bp (ALU115) and 247 bp (ALU247) PCR primer designs (below). Forward and reverse primers of ALU115 are indicated by green text, ALU247 primers by orange text. Brackets indicate the size of fragments (140-200 bp) generated by enzymatic apoptotic cleavage as compared to the total length of the Alu element. ALU115 primers amplify apoptotic and longer DNA fragments, while ALU247 primers only amplify sequences longer than apoptotic DNA.

The DNA fragmentation assay was performed on cfDNA abstract after quantification of extract total cfDNA concentration by RNaseP qPCR. Input was 50 pg, run in triplicate for both Alu fragment length amplifications against a five-point human genomic DNA standard curve. ALU115 and ALU247 amplifications were performed individually for each primer pair on a Roche Lightcycler 480 (LC280) using a shared TaqMan probe

(SEQ ID NO: 1)
(5′CCAGCCTGGCCAACATGGTG 3′).

The Lightcycler software was used to calculate a standard curve for the run by plotting the known DNA concentration of each standard dilution on the x-axis and the mean crossing point (Cp) value for those dilutions on the y-axis, also calculating the slope and amplification efficiency for each run. Patient sample cfDNA concentrations were individually determined by the Lightcycler software for the ALU115 and ALU247 amplifications using the calculated standard curve equation and the mean Cp as input. Results generated by the LC480 Abs Quant/2nd Derivative Max algorithm were captured in a report and used to determine Alu ratio by dividing the ALU247 concentration by the ALU115 concentration. Results are designed for use as a quality indicator of potentially significant leukocyte lysis/contamination of the patient sample that could potentially impact DF results and/or cause specimen rejection.

Analytical quality metrics developed to ensure validity of the fragmentation assay run include required ranges for ALU115 and ALU247 amplification efficiency, standard deviations of standard curve points, quantifications in pg/μ1 of low, medium, and high ALU115 and ALU247 controls, fragment ratios of specified standards, no template control (NTC) mean Cp, and specified standard Cp.

DNA fragmentation test—analytical validation methods: Analytical validation of the DNA fragmentation assay was designed to individually establish performance characteristics of the short (≥115 bp) and long (≥247 bp) fragment Alu amplification tests that together comprise the DNA fragmentation assay provided herein. LoB, LoD, LoQ, precision, accuracy, and linear range of the ALU115 and ALU247 amplifications were determined such that those characteristics in the resultant Alu ratio could be implied.

To support DNA fragmentation assay validation studies, as described above in Reference Materials, Covaris-sheared human buffy coat gDNA spiked back into aliquots of paired plasma was used to produce plasma samples at targeted long to short DNA fragmentation ratios of 0.2 to 0.5, yielding final actual ratios of 0.19 to 0.490. Additionally, for studies not linked to extraction, gDNA was introduced into 0.1×Tris-EDTA buffer. Samples with extraction were quantified by RNase P qPCR on a Roche LC480 prior to use in determinations of precision, DNA fragmentation assay LoB, LoD, LoQ, and linearity according to CLSI guidelines [1,2], see Results and Discussion. Specified acceptable ranges for individual standard curve amplification efficiencies and analytical measurement ranges were defined.

Interfering substances—analytical validation methods: Parallel sets of studies, essentially identical in design, were designed to assess the effects of potentially interfering substances on four individual aspects of the DNA fragmentation assay. qGT (DF determination). Ten substances were chosen for the study, including bilirubin, hemoglobin, EDTA, prednisone, tacrolimus, sirolimus, mycophenolate, cyclosporine A, triglycerides, and IVIg, these representing endogenous substances commonly elevated in plasma samples from heart transplant recipients as well as exogenous substances commonly introduced by standard medical therapies. Additionally, for extraction and genotyping validations, potential interference by two viruses, cytomegalovirus (CMV) and BK virus (BKV) was tested. Concentrations for each substance/virus were selected according to CLSI EP07-A2 [3], or previously published literature where appropriate. To summarize, tested substances and concentrations are as follows: bilirubin conjugate (20 mg/dl), hemoglobin (500 mg/dl), EDTA (1 ug/ml), prednisone (0.3 μg/ml), tacrolimus (40.2 ng/ml), sirolimus (12 ng/ml), mycophenolate mofetil (3.5 mcg/ml), cyclosporine A (400 ng/ml), triglycerides (30 mg/ml), IVIg (Gammaguard, 11 mg/ml), CMV virus (10,000 copies/ml), and BKV virus (10,000 copies/ml). Aliquots of individual patient and contrived patient samples prepared according to needs of each tested aspect of the assay (see sample preparation details below) were spiked with the potentially interfering substances, and, where indicated, the CMV and BK viruses, each in isolation. Aqueous or organic solvents required to dissolve a substance during their preparation (e.g., nuclease free water, ethanol, DMSO) were tested separately in the absence of that substance. Samples were then extracted in triplicate using the automated extraction procedure prior to processing through the intended workflow. Any samples not passing QC criteria during testing were removed from analysis. Passing results were analyzed using the statistical software package JMP, version 14 (SAS Institute, Inc., Cary, NC) using the Tukey-Kramer HSD test following one-way ANOVA testing to determine if the results from exposed samples deviated significantly from those of paired samples extracted and tested without spiked-in substance.

Sample preparation protocols and logistics unique to each interfering substance application are as follows: Interference studies for the DNA fragmentation assay analyses were performed in concert using three separate contrived human plasma samples prepared at total cfDNA concentrations targeted at 2 ng/ml, 25 ng/ml, and 50 ng/ml, each possessing a slightly different Alu ratio. All prepared samples were immediately frozen in 2 ml single use aliquots, then thawed and immediately spiked, extracted in triplicate and processed through the intended workflows and statistical analyses of results as outlined above.

Carryover/cross-contamination—analytical validation methods: Potential carryover/cross-contamination during extraction and downstream analytical workflows that could impact results of the DNA fragmentation analyses was assessed by testing high positive contrived samples (see Reference Materials) generated at −200 ng cfDNA/mL alongside negative (nuclease free water) samples in a 32-position checkboard pattern on the Tecan instrument across two independent runs. The extracted samples maintained the same sample positioning during subsequent DNA fragmentation testing (ALU115 and ALU247).

Validation of a Clinical DNA Fragmentation Assay for Quantitative Monitoring of Pre-Analytical Contamination of cfDNA with Leukocyte gDNA: To validate Alu ratio for use as a quantitative, clinical quality control measure to detect presence of significant leukocyte lysis in clinical samples submitted for cfDNA analysis, it was necessary to construct combinations of un-sheared and sheared gDNA to produce clinically relevant target Alu ratios in a range of ALU115 and ALU247 concentrations (1.56 pg/μ1 to 100 pg/μl, see Reference Materials). For some validation studies (e.g., ALU115 and ALU247 linearity, precision, LoQ) for which extraction was not required, human gDNA from a commercial vendor was used directly to make defined gDNA concentrations ranging from 0.25 pg/μ1 to 400 pg/μ1 in 0.1×TE Buffer. For other validation studies requiring DNA extraction, contrived samples prepared by spiking combinations of sheared and unsheared gDNA into aliquots of human plasma were employed (see Methods, Reference Materials).

Automated extraction of cfDNA from 4 ml volumes of contrived plasma samples prepared for this validation was performed on TECAN Freedom EVO 150 liquid handlers, followed by quantification by RNase P qPCR according to clinically validated protocols herein described.

Precision/LOB/LOD/LOQ, DNA Fragmentation Assay: Precision of ALU115 and ALU247 qPCR measurements was determined using commercially available gDNA diluted in 0.1×Tris-EDTA buffer to target concentrations of 100, 50, 25, 12.5, 6.25, 3.13, 1.56 pg/μl, each dilution tested for ALU115 and ALU247 amplification in duplicate wells per run, two runs per day for ten days, totaling 40 measurements for each dilution. % CV's ranged from a low of 11.4% at high target concentration (100 pg/μl, ALU 247) to a high of 24.8% at low target concentration (1.56 pg/μl, ALU247), see Tables 2 and 3.

TABLE 2
Precision Results, ALU115 qPCR

| Target cfDNA | | Measured Average | | | | |
|---|---|---|---|---|---|---|
| Concentration | Total | cfDNA Conc (pg/μl), | Standard | % CV | Lower | Upper |
| (pg/μl) | n | ALU115 qPCR | Deviation | Estimate | % CV | % CV |
| 100 | 40 | 99.1 | 14.7 | 14.8 | 12.1 | 19.1 |
| 50 | 40 | 46.9 | 6.00 | 12.8 | 10.4 | 16.5 |
| 25 | 40 | 22.9 | 3.54 | 15.5 | 12.6 | 20.0 |
| 12.5 | 40 | 12.8 | 1.50 | 11.7 | 9.6 | 15.1 |
| 6.25 | 40 | 6.57 | 1.22 | 18.7 | 15.2 | 24.2 |
| 3.13 | 40 | 3.18 | 0.45 | 14.1 | 11.5 | 18.2 |
| 1.56 | 40 | 1.68 | 0.33 | 19.9 | 16.2 | 25.8 |

TABLE 3
Precision Results, ALU247 qPCR

| Target cfDNA | | Measured Average | | | | |
|---|---|---|---|---|---|---|
| Concentration | Total | cfDNA Conc (pg/μl), | Standard | % CV | Lower | Upper |
| (pg/μl) | n | ALU247 qPCR | Deviation | Estimate | % CV | % CV |
| 100 | 40 | 96.5 | 10.97 | 11.4 | 9.3 | 14.6 |
| 50 | 40 | 47.9 | 8.51 | 17.8 | 14.5 | 23.0 |
| 25 | 40 | 23.1 | 3.00 | 13.0 | 10.6 | 16.8 |
| 12.5 | 40 | 12.7 | 1.74 | 13.7 | 11.2 | 17.7 |
| 6.25 | 40 | 6.37 | 0.96 | 15.0 | 12.2 | 19.4 |
| 3.13 | 40 | 3.19 | 0.62 | 19.3 | 15.7 | 25.1 |
| 1.56 | 40 | 1.69 | 0.42 | 24.8 | 20.1 | 32.4 |

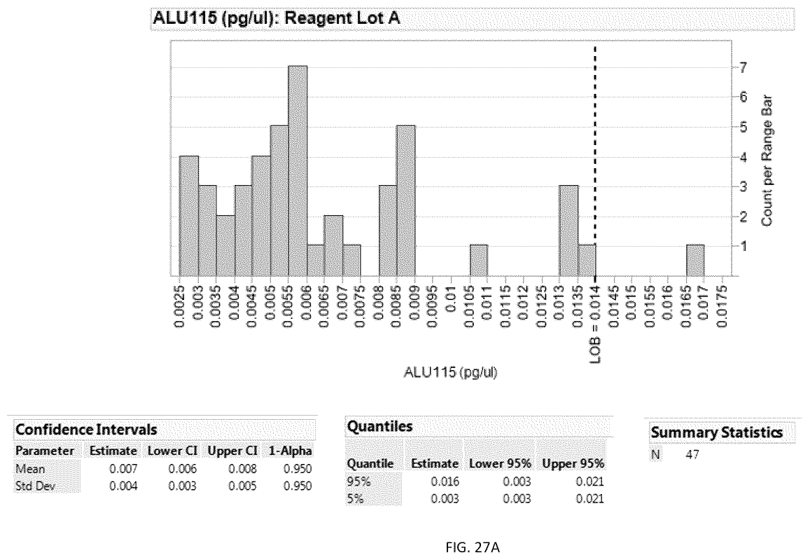

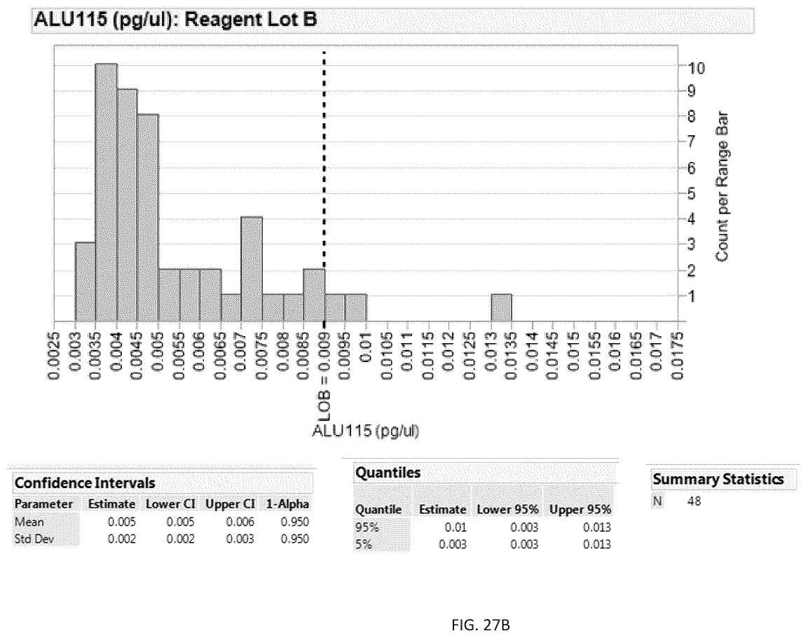

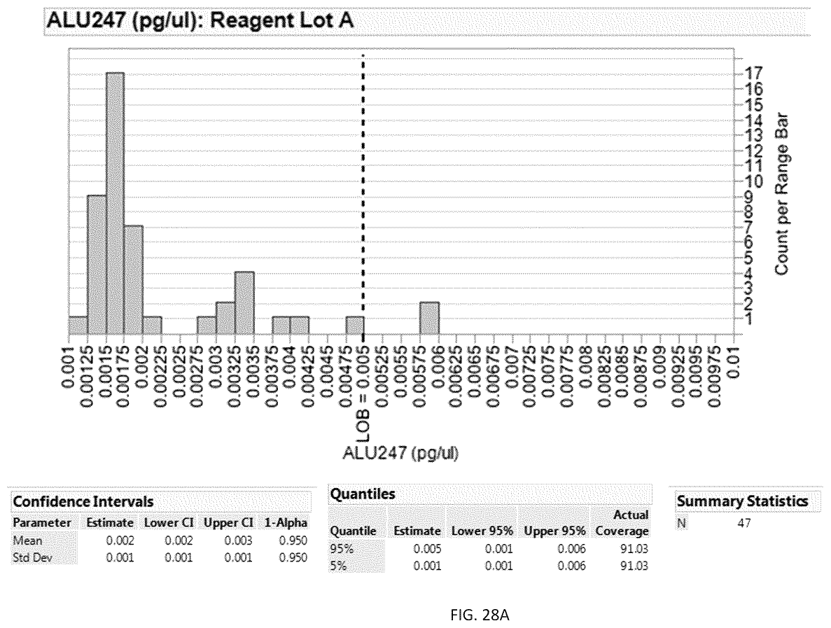

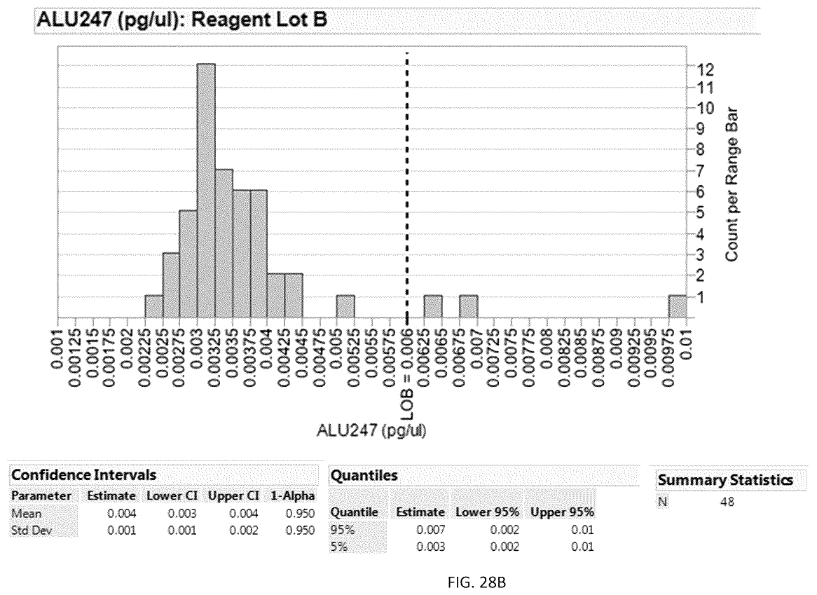

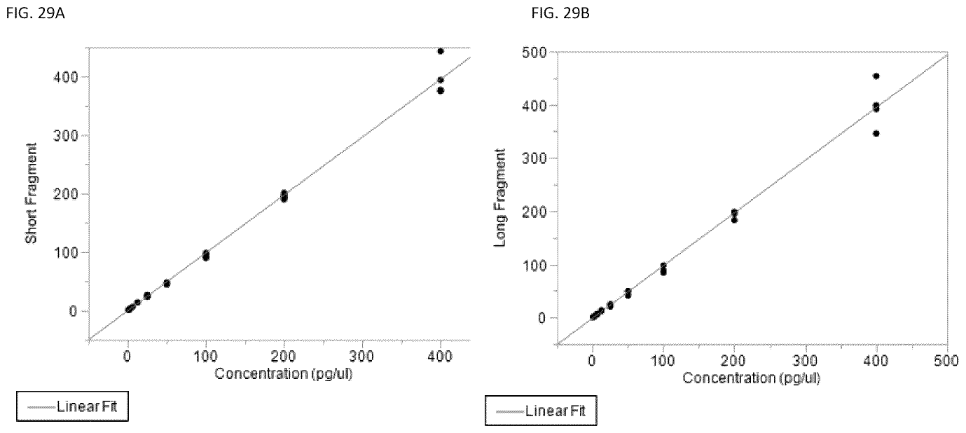

LoB values for the DNA fragmentation assay ALU115 and ALU247 fragment analyses were individually determined using 0.1×TE as the sample source. LoB values for ALU115 are shown in FIGS. 27A-27B and for ALU247 are shown in FIG. 28-28B. Twelve replicates were tested in eight runs performed twice per day across four days. Two lots of 0.1×TE were used for a total of 95 measurements (each) for the ALU115 and ALU247 amplifications. Resultant distributions of blanks for both ALU115 and ALU247 did not display a normal fit. The nonparametric option for obtaining LoB was used per CLSI EP17-A2 Evaluation of Detection Capability for Clinical Laboratory Management Measurement Procedures [1], assigning the final LoB for ALU115 as 0.014 pg/μ0.1 and the final LoB for ALU247 as 0.006 pg/μl, each representing the greater of the LoB values determined for the two tested 0.1×TE lots (see Table 4).

For determination of LoD values for the short and long fragment components of the DNA Fragmentation Assay, human gDNA (see Reference Materials) was diluted in 0.1×TE to concentrations of 4, 2, 1, 0.5 and 0.25 pg/μl, with each resultant sample tested in 5 wells per run and 2 runs per day for 4 days, yielding a total of 40 separate measurements collected across eight runs for each fragment length. Two lots of primers and probe were tested. LoD values for each assay (ALU115 and ALU247) were determined using the parametric approach as outlined in CLSI EP17-A2, pages 16-17 [1]. As the % CV for all of these low-level tested samples was <30%, statistics for the 0.25 pg/μl sample were used to perform LoD calculations. The resultant LoD is 0.122 pg/μl for ALU115 and 0.126 pg/μl for ALU247, representing the greater values determined for the two reagent lots (Table 4).

TABLE 4
Limit of Detection Results, ALU115 and ALU247.

| | Alu 115 Assay | | Alu 247 Assay | |
|---|---|---|---|---|
| | Reagent Lot A | Reagent Lot B | Reagent Lot A | Reagent Lot B |
| SD_{L} | 0.0654 | 0.0655 | 0.0728 | 0.053 |
| nᵢ | 40 | 40 | 40 | 40 |
| J | 1 | 1 | 1 | 1 |
| cₚ | 1.656 | 1.656 | 1.656 | 1.656 |
| L | 40 | 40 | 40 | 40 |
| LoB | 0.014 | 0.009 | 0.005 | 0.006 |
| LoD | 0.122 | 0.117 | 0.126 | 0.093 |

For determination of LoQ for the short and long fragment components of the DNA Fragmentation Assay, human gDNA prepared as described in Materials and Methods (Reference Materials) was diluted in 0.1×TE to concentrations of 4, 2, 1, 0.5 and 0.25 pg/μl. Each sample dilution was tested in 5 wells per run and 2 runs per day for four days, producing a total of 40 measurements collected across eight runs for each fragment length. Two lots of primers and probe were tested (Reagent Lot A and Reagent Lot B). LoQ for each assay was determined according guidelines outlined in CLSI EP17-A2 [1]. The LoQ for the short and long fragment assays were determined as follows using the data shown in Table 5. The mean and SD for the lowest level sample tested were calculated across all replicates for each reagent lot. The Bias was calculated by subtracting the assigned value (0.25 pg/μl) from the mean. The “TE” value was then determined using the equation TE=Bias+2*SD. Since the TE values calculated from both the short and long fragment data sets for the 0.25 pg/μl sample were <30%, the LoQ for both assays was determined to be 0.25 pg/μl.

TABLE 5
Limit of Quantitation Results, DNA Fragmentation Assay.

| | | ALU115 Assay | | | ALU247 Assay | | |
|---|---|---|---|---|---|---|---|
| Concentration | | Reagent | Reagent | | Reagent | Reagent | |
| (pg/μl) | | Lot A | Lot B | Pooled | Lot A | Lot B | Pooled |
| 4 | Average | 4.010 | 3.732 | 3.957 | 3.616 | 3.732 | 3.674 |
| | SD | 0.912 | 0.692 | 0.801 | 0.778 | 0.569 | 0.675 |
| | % CV | 23 | 18 | 20 | 22 | 15 | 18 |
| 2 | Average | 2.013 | 2.037 | 2.025 | 1.857 | 2.002 | 1.929 |
| | SD | 0.401 | 0.354 | 0.373 | 0.254 | 0.329 | 0.299 |
| | % CV | 20 | 17 | 18 | 14 | 16 | 15.5 |
| 1 | Average | 1.069 | 1.051 | 1.060 | 0.971 | 1.022 | 0.996 |
| | SD | 0.254 | 0.222 | 0.236 | 0.180 | 0.212 | 0.196 |
| | % CV | 24 | 21 | 22 | 19 | 21 | 19.6 |
| 0.5 | Average | 0.538 | 0.532 | 0.535 | 0.479 | 0.4992 | 0.489 |
| | SD | 0.121 | 0.114 | 0.116 | 0.079 | 0.097 | 0.088 |
| | % CV | 23 | 21 | 21.7 | 17 | 19 | 18 |
| 0.25 | Average | 0.286 | 0.283 | 0.285 | 0.255 | 0.264 | 0.259 |
| | SD | 0.065 | 0.066 | 0.065 | 0.073 | 0.053 | 0.063 |
| | % CV | 23 | 23 | 23 | 28.6 | 20 | 24.2 |

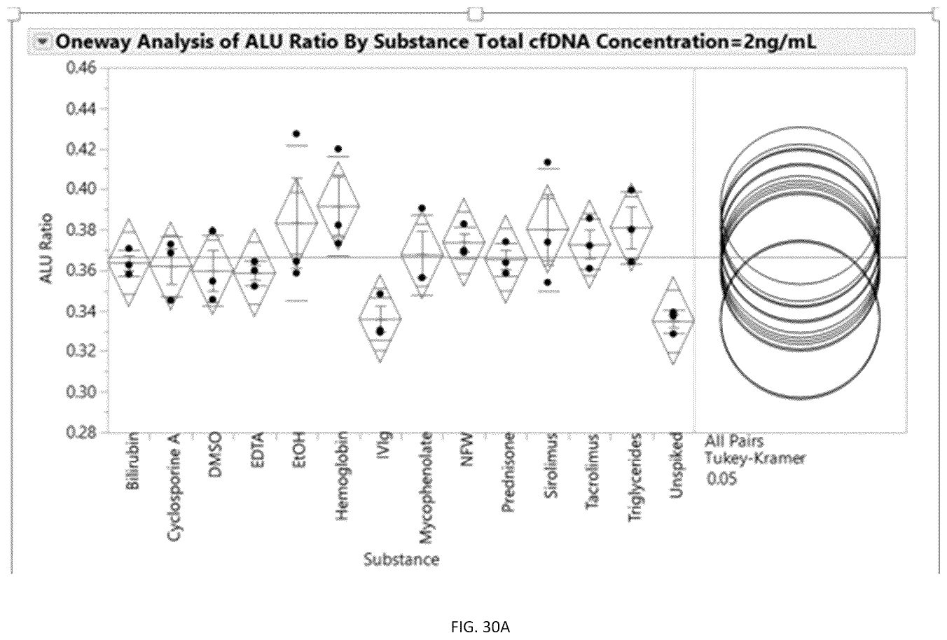

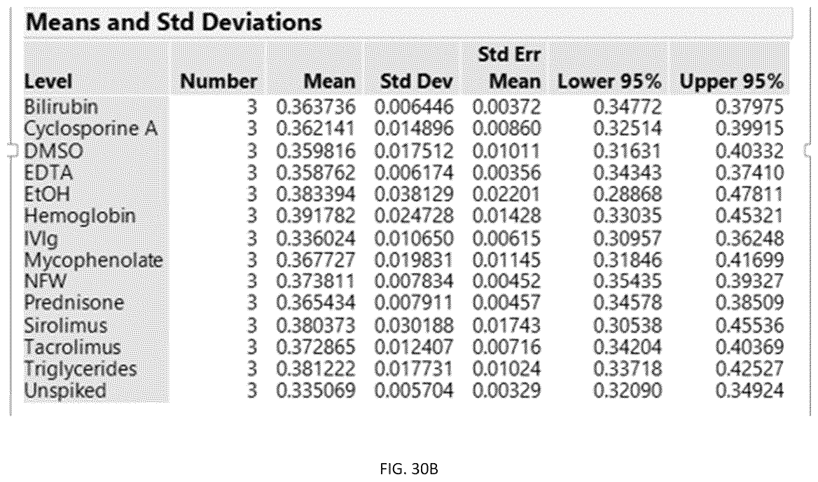

For linearity assessment of DNA Fragmentation Assay amplifications for ALU115 and ALU247, gDNA was diluted in 0.1×TE to the following concentrations: 400, 200, 100, 50, 25, 12.5, 6.25, 3.13, 1.56 and 0.78 pg/μl. Each linearity sample was tested in duplicate wells per run, two runs per day for one day for a total of four measurements collected across two runs. The resulting ALU115 and ALU247 amplification measurements, quantitated against a standard curve in units of pg/μ1 as described in Materials and Methods, was plotted against the theoretical concentration and assessed for linearity according to CLSI EP06-A [2] (FIG. 29A-B). ALU115 and ALU247 results were individually assessed for linear, second and third order polynomial fits within JMP (SAS Institute., Cary, NC). For both, resulting p-values were <0.05 for linear fit and >0.05 for second and third order polynomial fits; R-squared values for linear fit were >0.94 (ALU115) and >0.98 (ALU247), collectively indicating the results to be linear over the entire measured range of 0.78-400 pg/μl. (Table 6.)

TABLE 6
Linearity Fit Values, DNA Fragmentation Assay.

| Parameter | ALU115 Assay | ALU247 Assay |
|---|---|---|
| Dynamic Range (pg/μl) | 0.78-400 | 0.78-400 |
| Linear Fit p-value | <0.0001 | <0.0001 |
| Linear Fit R-squared | 0.9436 | 0.9890 |
| Second Order Polynomial Fit | 0.2498 | 0.2719 |
| p-value | | |
| Third Order Polynomial Fit p-value | 0.7215 | 0.6727 |

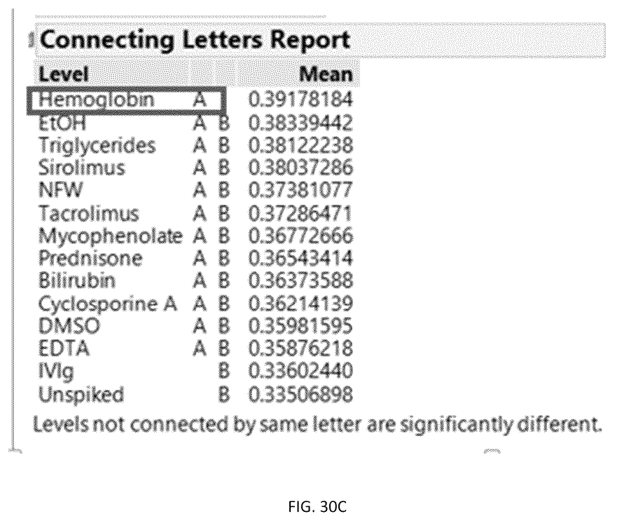

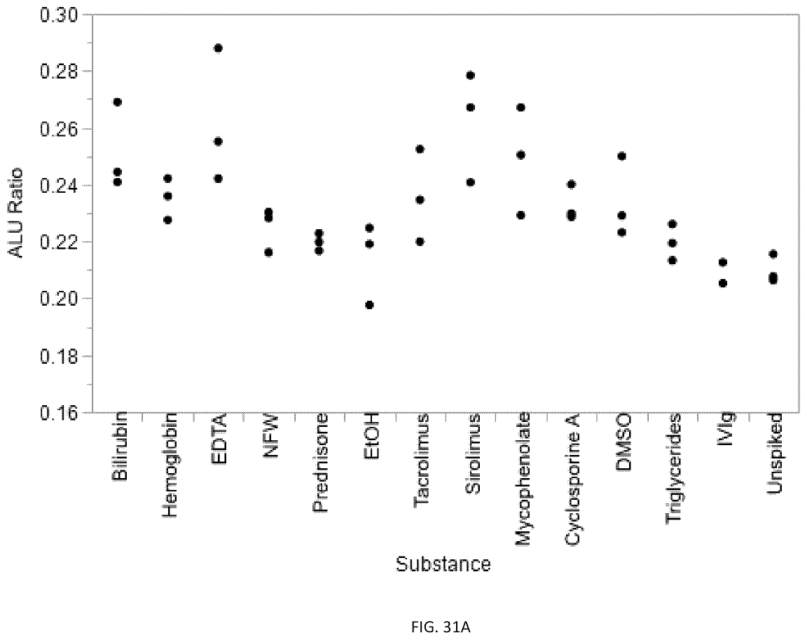

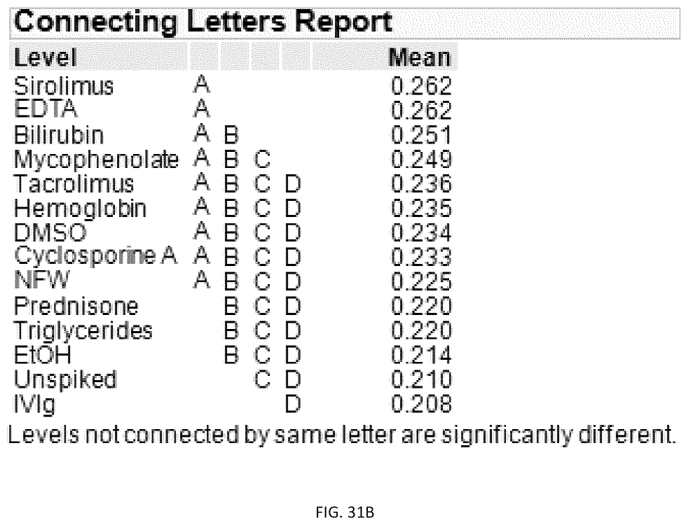

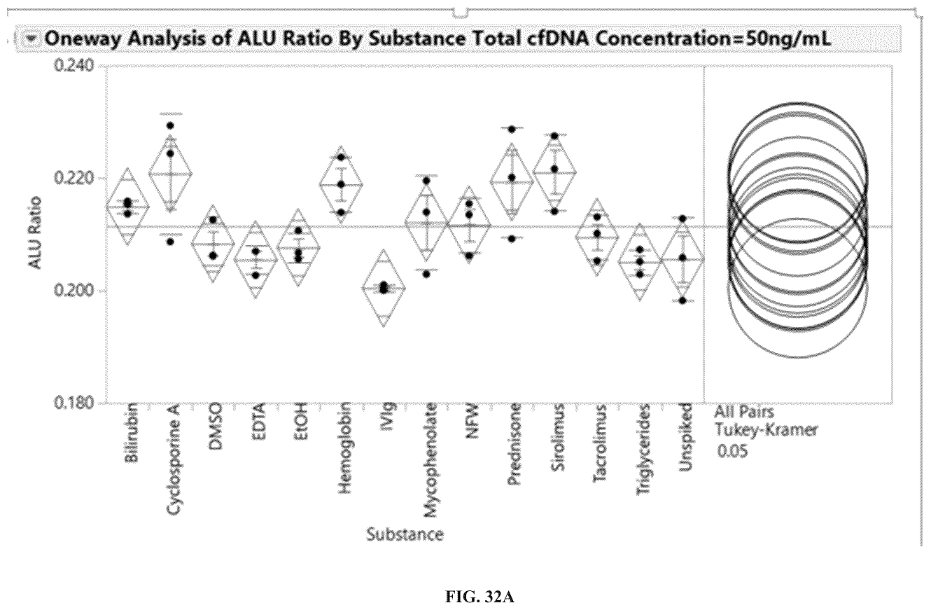

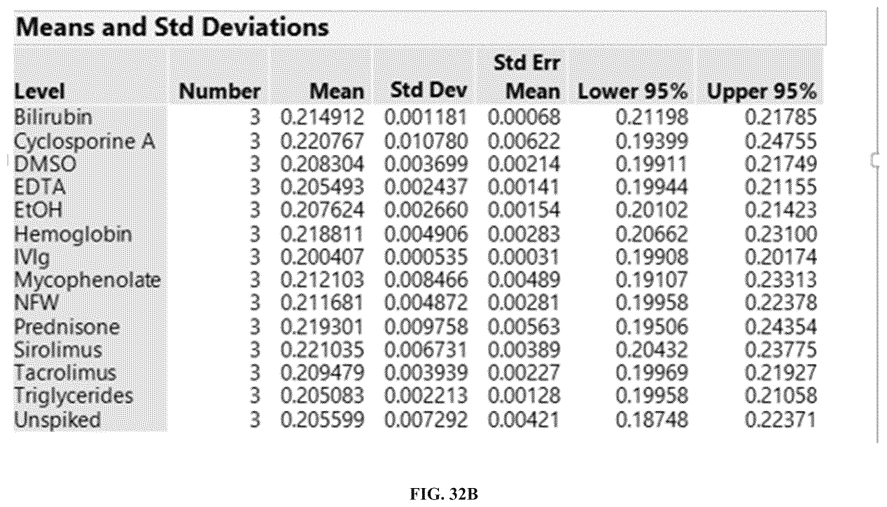

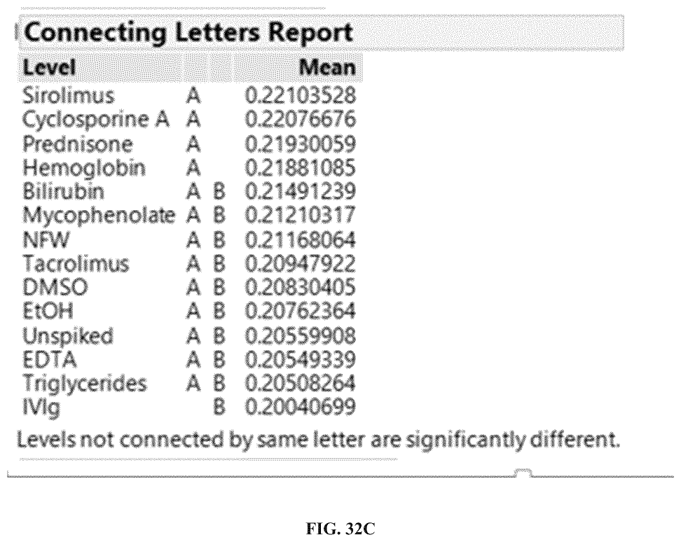

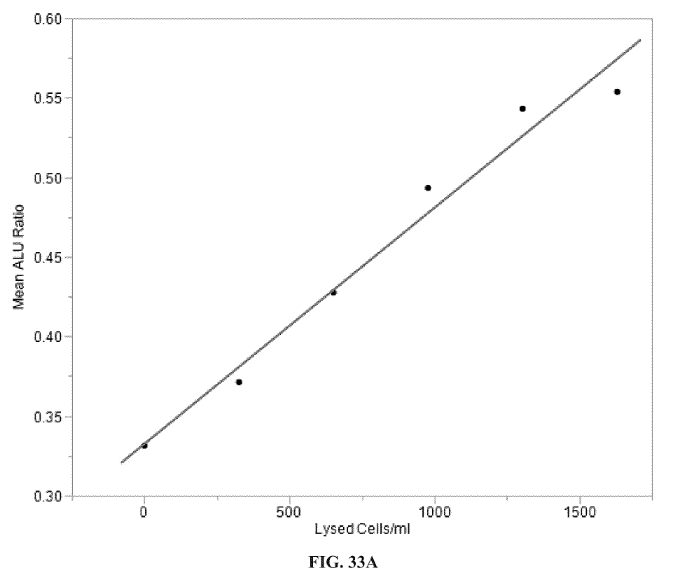

Interfering substances, DNA Fragmentation Assay: Effects of ten potentially interfering substances on the DNA fragmentation assay were assessed as described in Methods and Materials (see “Interfering Substance Assessment in Analytical Validations”) using three contrived human plasma samples prepared at three different TCF concentrations (2 ng/ml, 25 ng/ml, and 50 ng/ml) of variable Alu ratio. Alu ratio results were analyzed in JMP using the Tukey-Kramer HSD test following an ANOVA test to determine if the means were significantly different from samples extracted and tested without substance spiked-in. Results for 2 ng/ml are shown in FIG. 30A-30C. Results for 25 ng/ml are shown in FIG. 31A-31B. Results for 50 ng/ml are shown in FIG. 32A-C.

For all tested substances, no statistically significant differences compared to controls with diluting solvent without test substance spiked in were seen at any of the three cfDNA concentration. In addition, no statistically significant differences compared unspiked samples were seen for any substance at 50 ng/ml TCF. At 2 ng/ml cfDNA, a statistically significant, but not clinically significant, difference compared to the un-spiked control was seen for hemoglobin alone. At 25 ng/ml cfDNA, statistically, but not clinically significant differences compared to the unspiked control were seen for Sirolimus, EDTA, and bilirubin, but not compared to their respective solvent controls. No statistically significant differences were seen at 50 ng/ml TCF.

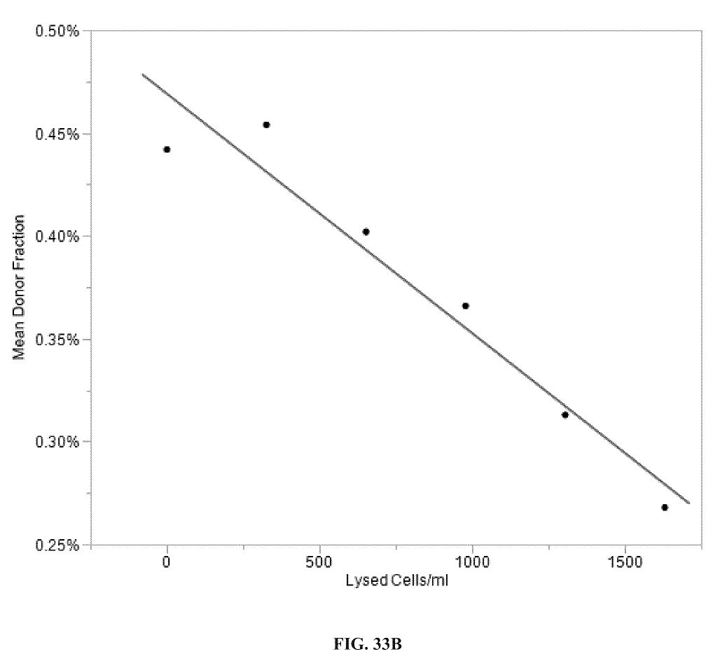

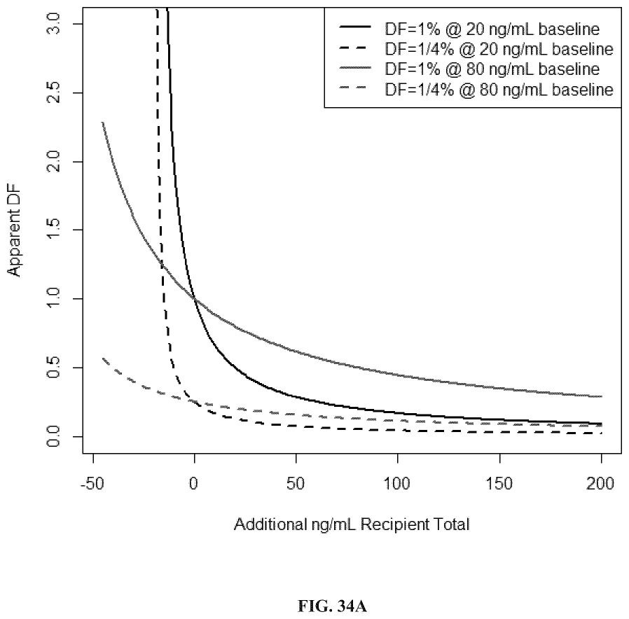

Detection of lysed leukocytes, DNA Fragmentation Assay, and effect on DF: The DNA Fragmentation Assay is designed to flag presence of excessive genomic DNA, such as genomic DNA released from leukocytes lysed during sample processing and shipping due to poor technique or extreme environmental exposures. To specifically test the quantitative performance characteristics of the DNA Fragmentation Assay in detecting leukocyte lysis, a leukocyte titration study was performed using multiple 1.5 ml aliquots of a contrived sample prepared by spiking plasma from one healthy “recipient” blood donor, sourced from blood bags provided by a commercial vendor, with “donor” plasma from a second healthy subject to give a theoretical DF of 0.4%. These “post-transplant” plasma aliquots were then spiked with specific numbers of leukocytes from the “recipient” donors buffy coat (enumerated by Cell Dyne cytometry), ranging from 0-2500 cells per 1.5 ml aliquot. After freezing at −80° C. to lyse the cells, the DNA fragmentation (ALU247/115) ratio was determined. Results are depicted graphically in FIG. 33A-B.

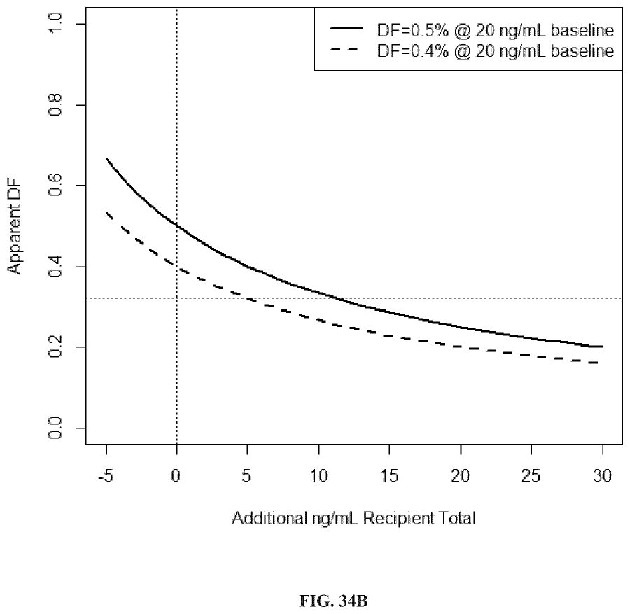

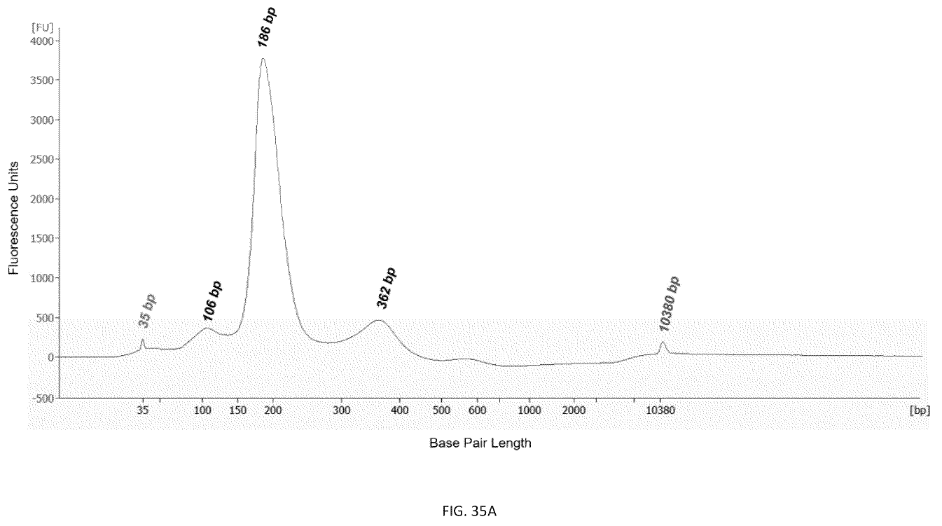

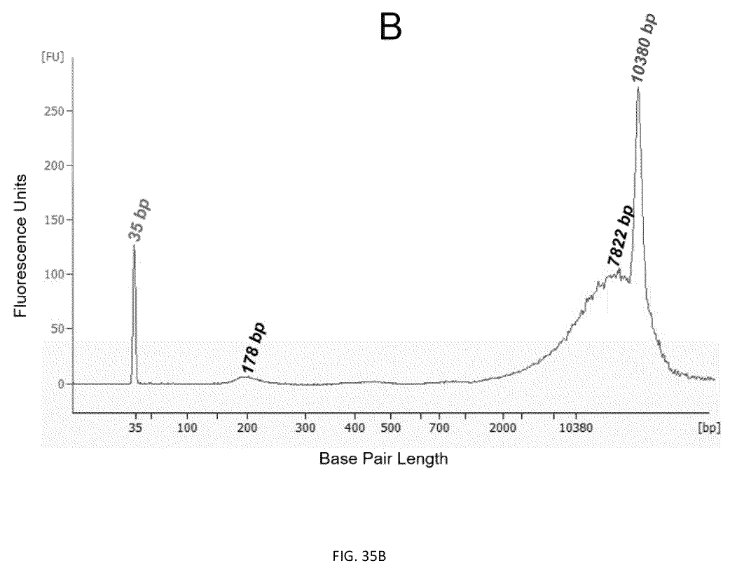

Results indicate that, within the tested range, Alu ratio and DF changes are linear relative to quantitated addition of lysed cells. These results further show that the DNA Fragmentation Assay can detect elevations of Alu ratio by DNA derived from presence of as few as 300 lysed cells/ml of plasma, this representing roughly 0.003% of the leukocytes in whole blood from which that plasma is purified, based on normal reference range clinical leukocyte counts. Within the tested range of leukocyte contamination/lysis (300-1667 lysed cells/ml plasma), DF can drop from roughly 0.45% to as low as 0.275%. Even low levels of leukocyte lysis or contamination during sample processing have potential to shift DF from the high probability rejection range into the low probability range (producing a false negative result) if not monitored by DNA fragmentation analysis. Plasma samples most sensitive to risk for potential production of a false negative DF result due to leukocyte lysis are those with low TCF concentration and relatively low DF. Mathematical modeling to estimate that sensitivity is shown in FIG. 34A-B.

Capillary electrophoresis (e.g., Agilent Bioanalyzer) electropherograms, as previously shown for a contrived cfDNA reference sample, can be used for clinical quality assurance purposes to evaluate DNA fragmentation independently of qPCR in unusual patient plasma extracts with cfDNA concentration high enough to reach threshold sensitivity for this methodology (roughly 600 ng/ml) without over utilizing limited patient material. Capillary electrophoresis was used to analyze the cfDNA fragmentation pattern of one such heart transplant patient (TCF concentration >6000 ng/ml and ALU115/247 ratio=0.19), comparing the results of the Alu PCR-based DNA fragmentation assay to those of this independent method. The unusually high cfDNA level, with low DF, in this patient stemmed from acute renal tubular injury at time of blood sample collection following an episode of cardiac arrest and resuscitation prior to eventual recovery. The sample was processed through using the standard, two low speed spin, plasma preparation protocol followed by automated extraction per Methods. It is informative to contrast the resultant electropherogram of the patient cfDNA extract collected by this protocol with one generated simultaneously for cfDNA extracted from plasma derived from a commercial normal donor blood lot shipped and received at TAI Diagnostics >24 hours after collection. It is clear that even for cfDNA from this heart transplant patient with very significant in vivo non-cardiac cellular injury, the DNA fragmentation pattern is compatible with apoptosis as the primary mechanism of cfDNA origin, whereas in the plasma commercially isolated and shipped without implementation of specific steps to avoid leukocyte lysis, the cfDNA population is largely long fragment, consistent with gDNA release from lysed leukocytes.

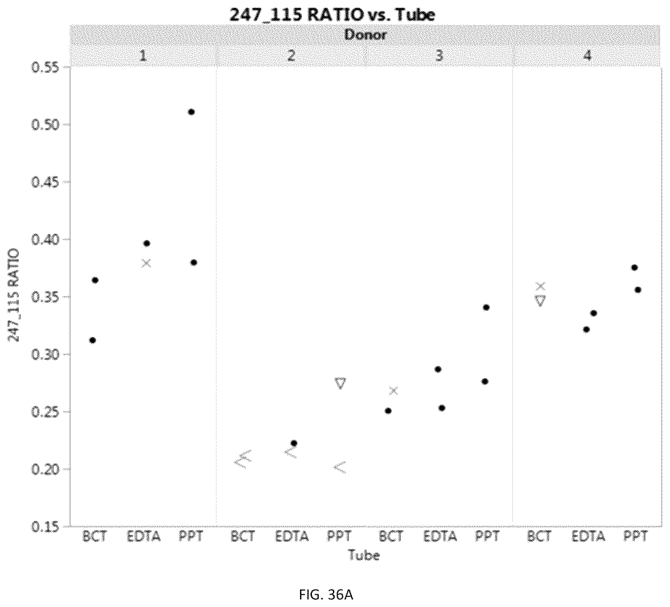

Bioanalyzer electropherograms of patient plasma cfDNA samples. (A) Patient sample collected and processed per TAI protocol shows predominant singlet and doublet apoptotic cfDNA peaks at 186 bp and 362 bp, respectively, without larger fragments produced by cellular lysis (FIG. 35A). Human sample procured and processed by a commercial vendor with delayed centrifugation (>24 hrs) shows a small peak at 178 bp (probably apoptotic) and a large, broad peak centered at 7822 bp, consistent with origin from leukocyte lysis (FIG. 35B). In both figures, sharp peaks at 35 bp and 10380 bp are internal kit markers.

Carryover/cross-contamination, DNA fragmentation Assay: Results of carryover/cross contamination analysis using contrived high positive samples generated at −200 ng cfDNA/mL alongside negative samples (nuclease free water) in a checkboard pattern maintained throughout TECAN extraction and DNA fragmentation analysis showed no evidence of carryover/cross contamination. Positive and negative extraction controls assured run validity, and no data was removed from analysis. All negative samples tested measured at or below the LoD for the ALU115 and ALU247 measurements (Table 7).

TABLE 7
ALU115 and ALU247 measurements

| ALU115 (pg/μl) | | | | ALU247 (pg/μl) | | | |
|---|---|---|---|---|---|---|---|
| 835 | 12150 | 0.121 | 13425 | 508 | 2238 | 0.0566 | 2550 |
| 0.0467 | 0.0866 | 13925 | 0.0877 | 0.0286 | 0.0513 | 2575 | 0.0526 |
| 790 | 14350 | 0.106 | 15075 | 449 | 2650 | 0.0526 | 2800 |
| 16400 | 0.0731 | 17425 | 0.0752 | 2650 | 0.0460 | 2925 | 0.0464 |
| 0.0673 | 13600 | 0.0718 | 17125 | 0.0399 | 2493 | 0.0443 | 2975 |
| 15875 | 0.0759 | 15875 | 0.0796 | 2700 | 0.0455 | 2875 | 0.0468 |
| 0.0726 | 13675 | 0.0702 | | 0.0427 | 2525 | 0.0462 | |
| 11250 | 0.0781 | 13775 | | 2328 | 0.0457 | 2675 | |

### REFERENCES

1. Clinical and Laboratory Standards Institute (CLSI). Evaluation of Detection Capability for Clinical Laboratory Measurement Procedures: Approved Guideline. CLSI document EP17-A2. ed 2. Wayne, PA, Clinical and Laboratory Standards Institute, 2012.
2. Clinical and Laboratory Standards Institute (CLSI). Evaluation of the Linearity of Quantitative Measurement Procedures: A Statistical Approach: Approved Guideline. CLSI document EP06-A. Wayne, PA, Clinical and Laboratory Standards Institute; 2003.
3. Clinical and Laboratory Standards Institute (CLSI). Interference Testing in Clinical Chemistry: Approved Guideline. CLSI document EP07-A2. Ed 2. Wayne, PA: Clinical and Laboratory Standards Institute; 2005.

### Example 9—Tubes and Cell Lysis

Measurement of non-self cell-free DNA fraction occurs after sample handling and shipment; therefore WBC lysis may have occurred. An amount of cell lysis can be determined using results from an Alu test as provided herein. The assays disclosed herein can also be used to determine if protocol differences increase cell lysis (e.g. variations in tubes, reagents, and/or sample handling procedures). For instance, several tubes have been tested to determine which tube might result in increased cell lysis.

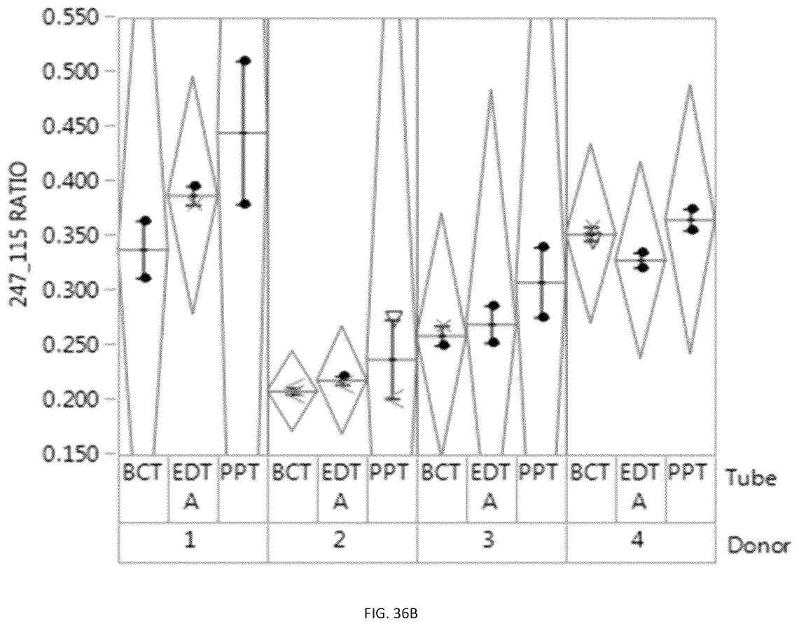

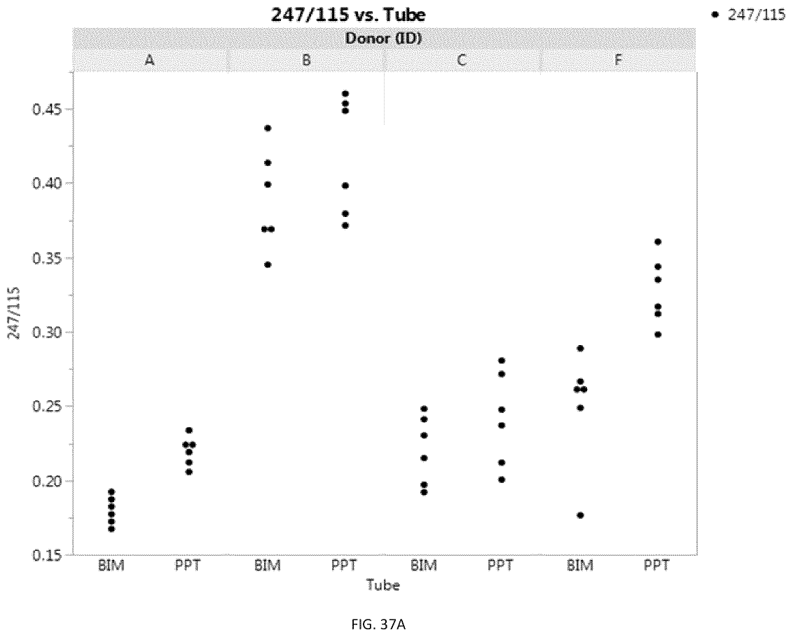

FIG. 36A-B shows a comparison of ALU247/115 ratios for samples collecting in BCT tubes, EDTA tubes, and PPT tubes from four different donors. Streck BCT tubes, EDTA tubes, and BD Bioscience's PPT tubes were compared.

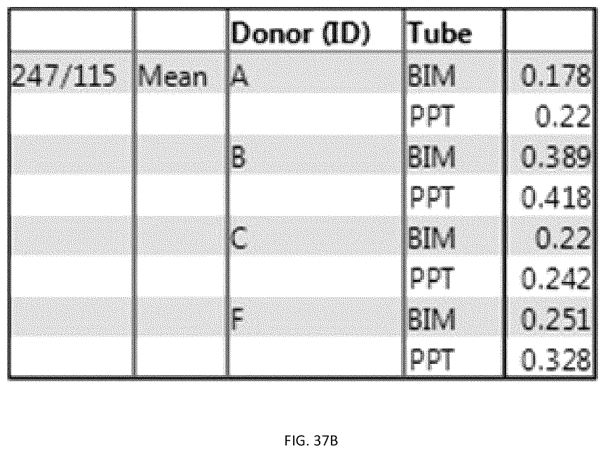

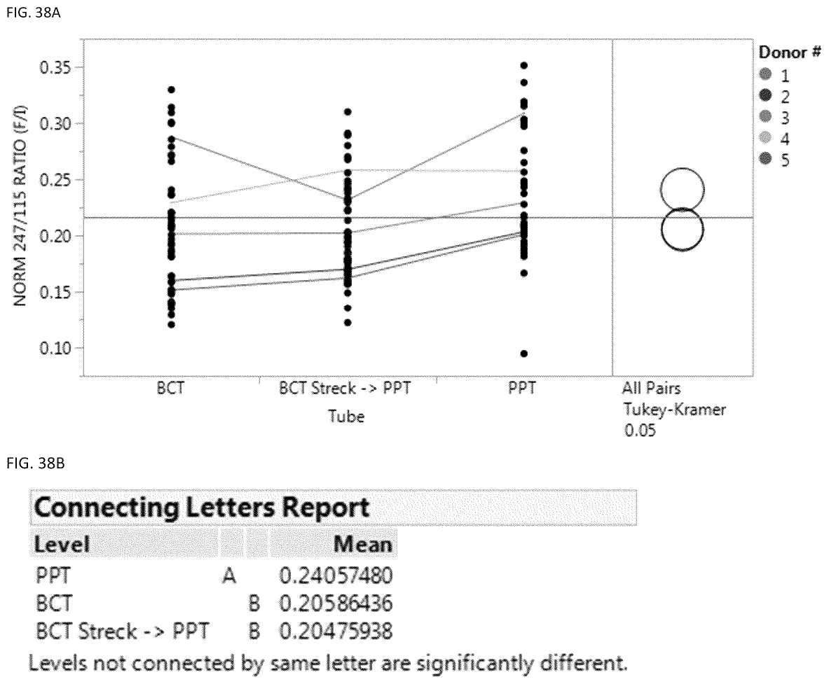

FIG. 37A-B shows a comparison of ALU247/115 ratios measured using SYBR for samples containing 50 pg input DNA. Biomatrica BIM cell preservation tubes and BD Bioscience's PPT tubes were compared. Results are shown in FIG. 37A and quantified in FIG. 37B.

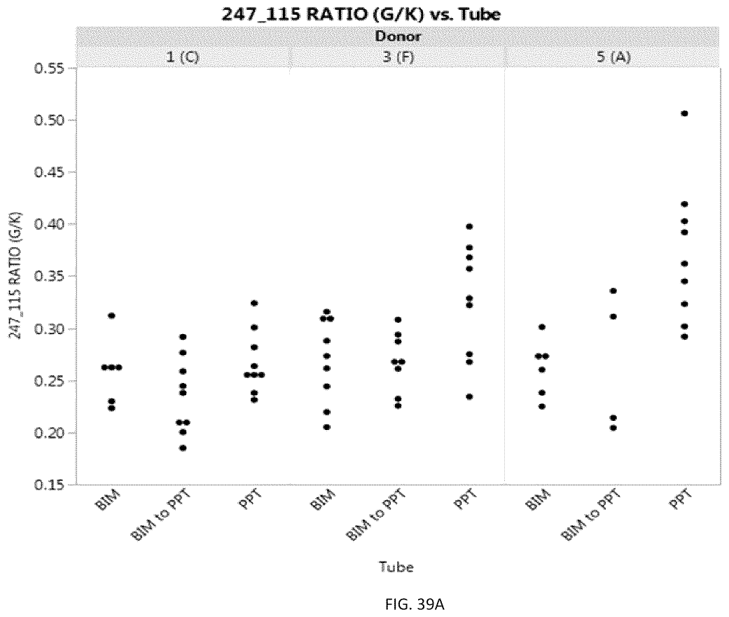

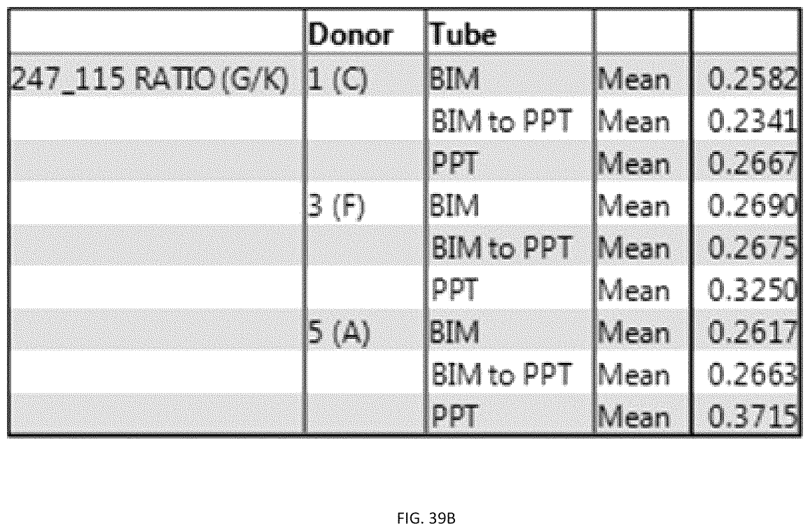

The increased Alu ratio findings were confirmed again in PPT tubes over BCT tubes in later experiments. In one experiment the cell preservation tube (BCT) against the PPT tube and a modified tube (BCT->PPT tube) were compared (FIG. 38A-B). In another experiment, the standard cell preservation tube (Streck BCT), a second cell preservation tube (Biomatrica BIM), and BD Bioscience's PPT tube which contains a separator plug, and a modification were compared. FIG. 39A shows a comparison of ALU247/115 ratios measured by SYBR for samples containing 50-70 pg input DNA in BIM tubes, PPT tubes, and a modified tube. Results are quantified in FIG. 39B.

The assays provided herein are superior to standard methods for analyzing cell lysis because it can be performed with a very small amount (picograms) of cfDNA which is already in limited amounts. Existing methods to detect cell lysis require 100-1000 times more cells or 10-fold more DNA.

1-27. (canceled)

28. A method for preparing a non-naturally occurring composition of amplified DNA from a liquid sample of a transplant recipient useful for selecting a suitable sample for quantifying donor-derived cell-free DNA, comprising:
(a) extracting cell-free DNA from the liquid sample of a transplant recipient;
(b) preparing a non-naturally occurring composition of amplified DNA by performing targeted amplification on the cell-free DNA to amplify a long Alu fragment that is 171-300 base pairs in length and a short Alu fragment that is 75-170 base pairs in length;
(c) analyzing the non-naturally occurring composition of amplified DNA obtained in (b) to quantify the long Alu fragment and the short Alu fragment; and
(d) selecting a sample of which a ratio of the quantified long Alu fragment over the quantified short Alu fragment is less than 0.3 and quantifying donor-derived cell-free DNA in the selected sample.

29. The method of claim 28, wherein the amplifications of the long Alu fragment and the short Alu fragment are performed using a forward primer and a reverse primer for the long Alu fragment, a forward primer and a reverse primer for the short Alu fragment, and one or more probes.

30. The method of claim 29, wherein the amplifications are performed using real time quantitative polymerase chain reaction (RT-qPCR).

31. The method of claim 29, wherein the amplifications are performed using one probe, wherein the probe is for the long Alu and the short Alu fragment.

32. The method of claim 31, wherein the probe comprises the nucleic acid sequence of SEQ ID NO: 1.

33. The method of claim 29, wherein the amplifications are performed using a first probe for the long Alu fragment and a second probe for the short Alu fragment.

34. The method of claim 33, wherein the first probe comprises the nucleic acid sequence of SEQ ID NO: 10 and the second probe comprises the nucleic acid sequence of SEQ ID NO: 13.

35. The method of claim 28, wherein the long Alu fragment is selected from ALU175, ALU224, ALU247, and ALU254.

36. The method of claim 28, wherein the short Alu fragment is selected from ALU115 and ALU79.

37. The method of claim 28, wherein the ratio is ALU247/ALU115.

38. The method of claim 28, wherein the method further comprises spiking the sample with a cell standard of at least 250 cells.

39. The method of claim 29, wherein the long Alu fragment and the short Alu fragment are quantified using a standard curve.

40. The method of claim 28, wherein the ratio of the quantified long Alu fragment over the quantified short Alu fragment of greater than 0.3 indicates the sample comprises contaminating DNA released from lysed leukocytes and is not suitable for quantifying donor-derived cell-free DNA.

41. The method of claim 28, wherein the ratio of the quantified long Alu fragment over the quantified short Alu fragment of greater than 0.5 indicates the sample comprises contaminating DNA released from lysed leukocytes and is not suitable for quantifying donor-derived cell-free DNA.

42. The method of claim 28, wherein the sample is a plasma sample, a serum sample, or a whole blood sample.


SEQUENCE LISTING (14 sequences)

SEQ ID NO: 1 - DNA, 20 bp, synthetic construct
  misc_feature 1..20: note = Synthetic
  source 1..20: mol_type = other DNA
ccagcctggccaacatggtg

SEQ ID NO: 2 - DNA, 20 bp, synthetic construct
  misc_feature 1..20: note = Synthetic
  source 1..20: mol_type = other DNA
cctgaggtcaggagttcgag

SEQ ID NO: 3 - DNA, 20 bp, synthetic construct
  misc_feature 1..20: note = Synthetic
  source 1..20: mol_type = other DNA
cccgagtagctgggattaca

SEQ ID NO: 4 - DNA, 19 bp, synthetic construct
  misc_feature 1..19: note = Synthetic
  source 1..19: mol_type = other DNA
gtggctcacgcctgtaatc

SEQ ID NO: 5 - DNA, 18 bp, synthetic construct
  misc_feature 1..18: note = Synthetic
  source 1..18: mol_type = other DNA
caggctggagtgcagtgg

SEQ ID NO: 6 - DNA, 20 bp, synthetic construct
  misc_feature 1..20: note = Synthetic
  source 1..20: mol_type = other DNA
tgtaatcccagctactcggg

SEQ ID NO: 7 - DNA, 18 bp, synthetic construct
  misc_feature 1..18: note = Synthetic
  source 1..18: mol_type = other DNA
ccactgcactccagcctg

SEQ ID NO: 8 - DNA, 18 bp, synthetic construct
  misc_feature 1..18: note = Synthetic
  source 1..18: mol_type = other DNA
cactttgggaggccgagg

SEQ ID NO: 9 - DNA, 25 bp, synthetic construct
  misc_feature 1..25: note = Synthetic
  source 1..25: mol_type = other DNA
ccactgcactccagcctgggcgaca

SEQ ID NO: 10 - DNA, 18 bp, synthetic construct
  misc_feature 1..18: note = Synthetic
  source 1..18: mol_type = other DNA
gtcaggagttcgaccagc

SEQ ID NO: 11 - DNA, 19 bp, synthetic construct
  misc_feature 1..19: note = Synthetic
  source 1..19: mol_type = other DNA
agctactcgggaggctgag

SEQ ID NO: 12 - DNA, 20 bp, synthetic construct
  misc_feature 1..20: note = Synthetic
  source 1..20: mol_type = other DNA
gcaggagaatcgcttgaacc

SEQ ID NO: 13 - DNA, 19 bp, synthetic construct
  misc_feature 1..19: note = Synthetic
  source 1..19: mol_type = other DNA
ggttgcagtgagccgagat

SEQ ID NO: 14 - DNA, 18 bp, synthetic construct
  misc_feature 1..18: note = Synthetic
  source 1..18: mol_type = other DNA
actccagcctgggcgaca